(19) United States
(12) Patent Application Publication
(10) Pub. No.: US 2026/0263213 A1
(43) Pub. Date: Sep. 10, 2026

(54) TEXTILES AND IMPLANTABLE MEDICAL DEVICES USING THE SAME

(71) Applicant: Edwards Lifesciences Corporation, Irvine, CA (US)

(72) Inventor: Jeffrey Michael Koslosky, Coto De Caza, CA (US)

(21) Appl. No.: 19/578,625

(22) Filed: Mar. 25, 2026

Related U.S. Application Data
(63) Continuation of application No. PCT/US2024/049390, filed on Oct. 1, 2024.
(60) Provisional application No. 63/587,408, filed on Oct. 2, 2023.

(51) Int. Cl. A61F 2/24

(52) U.S. Cl. CPC A61F 2/2412; A61F 2220/0008; A61F 2230/0063; A61F 2250/0017; A61F 2250/0036; A61F 2250/0069

(57) ABSTRACT

Disclosed herein is a 3-D unibody woven article comprising: a) a tubular member having a lumen; b) a cushioning member wherein a portion of the cushioning member comprises a central cavity housing a portion of the lumen; and c) a woven seal positioned at a distal end of the cushioning member.

### CROSS-REFERENCE TO RELATED APPLICATION

This application is a continuation of International Application No. PCT/US2024/049390, filed Oct. 1, 2024, which claims the benefit of U.S. Patent Application No. 63/587,408, filed Oct. 2, 2023, the entire disclosures all of which are incorporated herein by reference for all purposes.

### FIELD

The present application concerns aspects of textile materials that can be used in implantable medical devices. More specifically, the present application relates to woven unibody textile materials used as cushioning materials for implantable devices.

### BACKGROUND

The heart can suffer from various valvular diseases or malformations that result in significant malfunctioning of the heart and ultimately require the replacement of the native heart valve with an artificial valve. Human heart valves, which include the aortic, pulmonary, mitral, and tricuspid valves, function essentially as one-way valves operating in synchronization with the pumping heart. The valves allow blood to flow downstream but block blood from flowing upstream. Diseased heart valves exhibit impairments such as narrowing of the valve or regurgitation, which inhibits the valves'ability to control blood flow. Such impairments reduce the heart's blood-pumping efficiency and can be a debilitating and life-threatening condition. For example, valve insufficiency can lead to conditions such as heart hypertrophy and dilation of the ventricle. Thus, extensive efforts have been made to develop methods and apparatuses to repair or replace impaired heart valves.

Prostheses exist to correct problems associated with impaired heart valves. For example, mechanical and tissue-based heart valve prostheses can be used to replace impaired native heart valves. More recently, substantial effort has been dedicated to developing replacement heart valves, particularly tissue-based replacement heart valves that can be delivered with less trauma to the patient than through open-heart surgery. Replacement valves are being designed to be delivered through minimally invasive procedures and even percutaneous procedures. Such replacement valves often include a tissue-based valve body that is connected to an expandable frame that is then delivered to the native valve's annulus.

These replacement valves are often intended to at least partially block blood flow. However, a problem occurs when blood flows around the valve on the outside of the prosthesis. For example, in the context of replacement heart valves, paravalvular leakage has proven particularly challenging. An additional challenge relates to the ability of such prostheses to be secured relative to intra-luminal tissue, e.g., tissue within any body lumen or cavity, in an atraumatic manner. Further challenges arise when trying to controllably deliver and secure such prostheses in a location such as at a native mitral valve. These replacement valves are often intended to at least partially block blood flow.

Because of the drawbacks associated with conventional open-heart surgery, percutaneous and minimally-invasive surgical approaches are garnering intense attention. In one technique, a prosthetic valve is configured to be implanted in a much less invasive procedure by way of catheterization. For instance, U.S. Pat. Nos. 10,716,664, 9,681,951, 10,583,000, 11,224,507, and International Patent Publication Nos. WO2018017886 and WO2019/169087, which are incorporated herein by reference in their entireties for all purposes, describe heart valve prostheses for replacing native valves, including a self-expanding frame with a plurality of anchoring members that are designed to be deployed within a body cavity and prevent axial flow of fluid around an exterior of the prosthesis.

However, problems can still occur. For example, some challenges related to the ability of such prostheses to be secured relative to intra-luminal tissue, e.g., tissue within any body lumen or cavity, in an atraumatic manner. Further challenges arise when trying to controllably deliver and secure such prostheses in the desired location.

These needs and others are at least partially satisfied by the present disclosure.

### SUMMARY

Some of the aspects of the present disclosure relate to textiles. Some aspects relate to woven articled defined by a proximal end and a distal end, wherein the woven article is a 3-D unibody article and comprises: a) a tubular member having a lumen disposed between a first open end of the tubular member and a second end of the tubular member; wherein the tubular member has a first thickness, wherein the first open end defines the proximal end of the woven article; wherein the lumen has a diameter d₁ and a length l₁; b) a cushioning member having a second thickness, wherein an outer surface of the tubular member seamlessly transitions into an outer surface of the cushioning member at a length l₂ from the first open end of the tubular member, thereby forming a proximal end of the cushioning member, wherein l₂ is less than l₁, wherein a portion of the cushioning member comprises a central cavity housing a portion of the lumen having a length l₃, such that the second end of the tubular member is positioned within the central cavity of cushioning member; and c) a woven seal positioned at a distal end of the cushioning member.

Yet, further disclosed herein are aspects wherein the tubular member has a plain weave pattern, a twill weave, a satin weave, any derivative, or any combination thereof. Still further disclosed herein are aspects wherein the cushioning member has a 3-D honeycomb pattern having a plurality of repeating cell units.

Also disclosed herein are aspects wherein the woven article further comprises a covering member having a third thickness and is defined by an inner surface and an outer surface and having a proximal end and a distal end, wherein the proximal end of the covering member is coupled to the woven seal and wherein the distal end is open.

Also disclosed herein are aspects wherein the woven article is configured to be assembled in an implantable medical device comprising: one or more anchors, wherein each of the one or more anchors is configured to anchor the implantable medical device into a patient's natural anatomy.

In some aspects, the woven article is configured to receive an anchor of the one or more anchors through the first open end of the tubular member such that the anchor is positioned within the lumen of the tubular member, such that the second end of the tubular member substantially prevents a further anchor's movement within the article.

In other aspects disclosed herein is an implantable medical device comprising one or more anchors, wherein each of the one or more anchors is configured to anchor the implantable medical device to a portion of a patient's natural anatomy, wherein each of the anchors is positioned within any of the disclosed herein the woven articles.

Yet some aspects described herein, relate to implantable device comprising a self-expanding frame having an upper region, an intermediate region and a lower region, wherein the frame is configured to radially expand and contract for deployment within a body cavity, and wherein the self-expanding frame comprises one or more anchors disposed along the lower region; wherein each of the one or more anchors is configured to anchor the implantable medical device to a portion of a patient's natural anatomy, and wherein at least a portion of the one or more anchors is positioned within a woven article; wherein the woven article comprises: a tubular member having a lumen disposed between a first open end of the tubular member and a second end of the tubular member; wherein the tubular member has a first thickness, wherein the first open end defines the proximal end of the woven article; wherein the lumen has a diameter d₁ and a length l₁; a cushioning member having a second thickness, wherein an outer surface of the tubular member seamlessly transitions into an outer surface of the cushioning member at a length l₂ from the first open end of the tubular member, thereby forming a proximal end of the cushioning member, wherein l₂ is less than l₁, wherein a portion of the cushioning member comprises a central cavity housing a portion of the lumen having a length l₃, such that the second end of the tubular member is positioned within the central cavity of cushioning member; and a woven seal positioned at a distal end of the cushioning member.

Some aspects disclosed herein relate to a method comprising weaving any of the disclosed herein woven articles.

Some aspects disclosed herein relate to a method of making any of the disclosed herein implantable medical devices, wherein the method comprises: a) providing a device comprising one or more anchors, wherein each of the one or more anchors is configured to anchor the implantable medical device to a portion of a patient's natural anatomy, and b) inserting each anchor into any of the disclosed herein woven articles.

Also disclosed herein are methods comprising: deploying a prosthetic valve to a native valve of a patient's heart, the prosthetic valve comprising: a plurality of prosthetic valve leaflets, one or more anchors coupled to the plurality of prosthetic valve leaflets, and each configured to anchor to a portion of the patient's heart, wherein each of the one or more anchors is inserted into any of the disclosed herein woven articles.

Additional aspects of the disclosure will be set forth, in part, in the detailed description, figures, and claims that follow, and in part, will be derived from the detailed description or can be learned by practice of the disclosure. It is to be understood that both the foregoing general description and the following detailed description are exemplary and explanatory only and are not restrictive of the disclosure as disclosed.

### BRIEF DESCRIPTION OF THE DRAWINGS

FIG. 1 shows a frame of an exemplary implantable medical device according to one aspect.

FIGS. 2A-2B show an exemplary woven article positioned on an exemplary implantable medical device in one aspect (FIG. 2A) and a close-up view of the exemplary woven article (FIG. 2B).

FIG. 3 shows a schematic construction of an exemplary woven article according to one aspect.

FIG. 4 shows a formed exemplary woven article according to one aspect.

FIG. 5 shows a schematic construction of an exemplary woven article according to an additional aspect.

FIGS. 6A-6C show a formed exemplary woven article according to the schematic of FIG. 5.

FIG. 7 shows a cross-sectional schematic view of an exemplary assembled woven article coupled to an anchor according to one aspect.

FIG. 8A shows a perspective view of an exemplary implantable medical device according to one aspect.

FIG. 8B shows a bottom view of the exemplary implantable medical device shown in FIG. 8A according to one aspect.

FIG. 9A shows an exemplary woven article, as shown in FIG. 4, being coupled to an anchor of an exemplary implantable medical device according to one aspect.

FIG. 9B shows an exemplary woven article, as shown in FIGS. 6A-6C being coupled to an anchor of an exemplary implantable medical device according to one aspect.

FIG. 10 shows a view of a surface of a cushioning member according to one aspect.

FIG. 11 shows a perspective view of a representation of a surface of a weave pattern of a cushioning member according to one aspect.

FIG. 12A shows a photograph of a cushioning member as woven.

FIG. 12B shows a photograph of the cushioning member shown in FIG. 12A following an exemplary treatment according to one aspect.

FIG. 13 illustrates a perspective view of a delivery apparatus according to one aspect.

FIG. 14A illustrates a cross-sectional view of an implant retention area of a delivery apparatus according to one aspect.

FIG. 14B illustrates a cross-sectional view of the implant retention area shown in FIG. 14A according to one aspect.

FIG. 15 illustrates a delivery apparatus approaching a mitral valve according to one aspect.

FIG. 16A illustrates a side perspective view of a valve being deployed from a delivery apparatus according to one aspect.

FIG. 16B illustrates a side perspective view of a valve being deployed from a delivery apparatus according to one aspect.

FIG. 16C illustrates a side perspective view of a valve being deployed from a delivery apparatus according to one aspect.

FIG. 17 illustrates a schematic view of a valve deployed to a mitral valve according to one aspect.

### DETAILED DESCRIPTION

The present disclosure can be understood more readily by reference to the following detailed description, examples, drawings, and claims, and their previous and following description. However, before the present articles, systems, and/or methods are disclosed and described, it is to be understood that this disclosure is not limited to the specific or exemplary aspects of articles, systems, and/or methods disclosed unless otherwise specified, as such can, of course, vary. It is also to be understood that the terminology used herein is for the purpose of describing particular aspects only and is not intended to be limiting.

The following description of the disclosure is provided as an enabling teaching of the disclosure in its best, currently known aspect. To this end, those skilled in the relevant art will recognize and appreciate that many changes can be made to the various aspects of the disclosure described herein while still obtaining the beneficial results of the present disclosure. It will also be apparent that some of the desired benefits of the present disclosure can be obtained by selecting some of the features of the present disclosure without utilizing other features. Accordingly, those of ordinary skill in the pertinent art will recognize that many modifications and adaptations to the present disclosure are possible and can even be desirable in certain circumstances and are a part of the present disclosure. Thus, the following description is again provided as illustrative of the principles of the present disclosure and not in limitation thereof.

### Definitions

As used in this application and in the claims, the singular forms “a,” “an,” and “the” include the plural forms unless the context clearly dictates otherwise. Thus, for example, reference to a “yarn” includes aspects having two or more such yarns unless the context clearly indicates otherwise.

It is also to be understood that the terminology used herein is for the purpose of describing particular aspects only and is not intended to be limiting. As used in the specification and in the claims, the term “comprising” can include the aspects “consisting of” and “consisting essentially of.” Additionally, the term “includes” means “comprises.”

For the terms “for example,” “exemplary,” and “such as,” and grammatical equivalences thereof, the phrase “and without limitation” is understood to follow unless explicitly stated otherwise.

Ranges can be expressed herein as from “about” one particular value and/or to “about” another particular value. When such a range is expressed, another aspect includes from the one particular value and/or to the other particular value. Similarly, when values are expressed as approximations, by use of the antecedent “about,” it will be understood that the particular value forms another aspect. It should be further understood that the endpoints of each of the ranges are significant both in relation to the other endpoint and independently of the other endpoint. Unless stated otherwise, the term “about” means within 5% (e.g., within 2% or 1%) of the particular value modified by the term “about.” It is further understood that if the term “about 1” is disclosed, then also term “1” is disclosed.

Throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, a description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, 6 and any whole and partial increments therebetween. This applies regardless of the breadth of the range.

As used herein, the terms “optional” or “optionally” mean that the subsequently described event or circumstance can or cannot occur and that the description includes instances where said event or circumstance occurs and instances where it does not.

Further, the terms “coupled” and “associated” generally mean electrically, electromagnetically, and/or physically (e.g., mechanically or chemically) coupled or linked and do not exclude the presence of intermediate elements between the coupled or associated items.

It will be understood that when an element is referred to as being “connected” or “coupled” to another element, it can be directly connected or coupled to the other element, or intervening elements can be present. In contrast, when an element is referred to as being “directly connected” or “directly coupled” to another element, there are no intervening elements present. Other words used to describe the relationship between elements or layers should be interpreted in a like fashion (e.g., “between” versus “directly between,” “adjacent” versus “directly adjacent,” “on” versus “directly on”).

It will be understood that although the terms “first,” “second,” etc., can be used herein to describe various elements, components, regions, layers and/or sections. These elements, components, regions, layers, and/or sections should not be limited by these terms. These terms are only used to distinguish one element, component, region, layer, or section from another element, component, region, layer, or a section. Thus, a first element, component, region, layer, or section discussed below could be termed a second element, component, region, layer, or section without departing from the teachings of example aspects.

Spatially relative terms, such as,” “beneath,” “below,” “lower,” “above,” “upper,” “upward,” “downward,” “top,” “bottom,” and the like, can be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, the term “below” can encompass both an orientation of above and below. The device can be otherwise oriented (rotated 90 degrees or at other orientations), and the spatially relative descriptors used herein are interpreted accordingly.

Terms such as “proximal,” “distal,” “radially outward,” “radially inward,” “outer,” “inner,” and “side” describe the orientation and/or location of portions of the components or elements within a consistent but arbitrary frame of reference which is made clear by reference to the text and the associated drawings describing the components or elements under discussion. Such terminology can include the words specifically mentioned above, derivatives thereof, and words of similar import. Similarly, the terms “first,” “second,” and other such numerical terms referring to structures neither imply a sequence nor order unless clearly indicated by the context.

As used herein, the term “and/or” includes any and all combinations of one or more of the associated listed items. It is also understood that the term “and/or” includes where one or another of the associated listed items is present, and the aspects where both of the associated listed items are present or any combinations of the associated listed items are present.

As used herein, the term or phrase “effective,” “effective amount,” or “conditions effective to” refers to such amount or condition that is capable of performing the function or property for which an effective amount or condition is expressed. As will be pointed out below, the exact amount or particular condition required will vary from one aspect to another, depending on recognized variables such as the materials employed and the processing conditions observed. Thus, it is not always possible to specify an exact “effective amount” or “condition effective to.” However, it should be understood that an appropriate effective amount will be readily determined by one of ordinary skill in the art using only routine experimentation.

The terms “fiber” or “filament” can be used interchangeably.

The terms “fiber” (or “filament”) and “material comprising a plurality of fibers (filaments)” are used herein according to their broad and ordinary meanings and can refer to any type of natural or synthetic substance or material that is significantly longer than it is wide, including any elongate or relatively fine, slender, and/or threadlike piece, filament, cord, yarn, plie, strand, line, string, or portion thereof. Furthermore, “fiber” or “material comprising a plurality of fibers” can refer to a single filament or collectively to a plurality of filaments. Examples of material comprising a plurality of fibers in accordance with aspects of the present disclosure include, but are not limited to, any type of cloth, fabric, or textile. It is understood that in certain unlimiting aspects, the term “material comprising a plurality of fibers” can refer to cloth, fabric, textile, or interlocking-fiber material that can cover or form certain features of the disclosed devices.

The term “fiber,” as used herein, includes fibers of extreme or indefinite length (e.g., filaments) and fibers of short length (e.g., staple fibers).

As used herein, the term “substantially” means that the subsequently described event or circumstance completely occurs or that the subsequently described event or circumstance generally, typically, or approximately occurs.

Still further, the term “substantially” can, in some aspects, refer to at least about 90%, at least about 91%, at least about 92%, at least about 93%, at least about 94%, at least about 95%, at least about 96%, at least about 97%, at least about 98%, at least about 99%, or about 100% of the stated property, component, composition, or other condition for which substantially is used to characterize or otherwise quantify an amount.

As used herein, the term “substantially,” in, for example, the context “substantially identical” or “substantially similar,” refers to a method or a system, or a component that is at least about 90%, at least about 91%, at least about 92%, at least about 93%, at least about 94%, at least about 95%, at least about 96%, at least about 97%, at least about 98%, at least about 99%, or about 100% by similar to the method, system, or the component it is compared to.

As disclosed herein, it is understood that when the reference is done to a thickness of the indicated member, the referred thickness is a thickness of the member wall.

Although the operations of exemplary aspects of the disclosed method can be described in a particular, sequential order for convenient presentation, it should be understood that disclosed aspects can encompass an order of operations other than the particular, sequential order disclosed. For example, operations described sequentially can, in some cases, be rearranged or performed concurrently. Further, descriptions and disclosures provided in association with one particular aspect are not limited to that aspect and can be applied to any aspect disclosed.

Moreover, for the sake of simplicity, the attached figures cannot show the various ways (readily discernable, based on this disclosure, by one of ordinary skill in the art) in which the disclosed system, method, and apparatus can be used in combination with other systems, methods, and apparatuses. Additionally, the description sometimes uses terms such as “produce” and “provide” to describe the disclosed method. These terms are high-level abstractions of the actual operations that can be performed. The actual operations that correspond to these terms can vary depending on the particular implementation and are, based on this disclosure, readily discernible by one of ordinary skill in the art.

### Woven Article

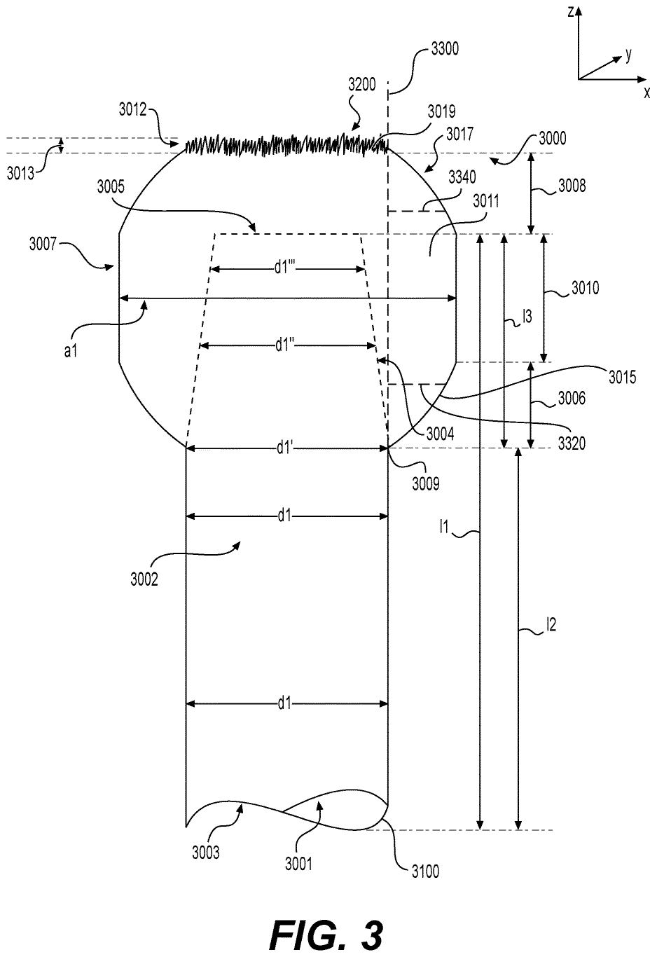

The disclosed herein woven article is a 3-D unibody article, components (or members) of which are constructed together during the weaving process. It is understood that depending on the desired application, the article can comprise any or some of the disclosed herein members. An exemplary woven 3-D unibody article is shown in FIG. 3.

Disclosed herein is a woven article defined by a proximal end 3100 and a distal end 3200. In some aspects, the disclosed woven article 3000 comprises a tubular member 3002 having a lumen 3001 disposed between a first open end 3003 of the tubular member 3002 and a second end 3005 of the tubular member. In some aspects, the first open end 3003 defines the proximal end 3100 of the woven article. In some aspects, the tubular member has a first thickness. In some further aspects, the lumen has a diameter d₁ and a length l₁.

In still further aspects, the woven article 3000 further comprises a cushioning member 3007 having a second thickness, wherein an outer surface of the tubular member seamlessly transitions into an outer surface of the cushioning member at a length l₂ from the first open end of the tubular member, thereby forming a proximal end 3009 of the cushioning member, wherein l₂ is less than l₁. In still further aspects, a portion of the cushioning member comprises a central cavity 3011 housing a portion 3004 of the lumen having a length l₃, such that the second end 3005 of the tubular member is positioned within the central cavity of the cushioning member. It is understood that the length l₃ is equal to l₁−l₂. In aspects disclosed herein, the outer surface of the cushioning member is formed by transitioning the outer surface of the tubular member, while an inner surface of the cushioning member defines at least a portion of the central cavity of the cushioning member.

In aspects disclosed herein, the first thickness of the tubular member can be about 0.06 mm to about 0.15 mm, including exemplary values of about 0.07 mm, about 0.08 mm, about 0.09 mm, about 0.1 mm, about 0.11 mm, about 0.12 mm, about 0.13 mm, and about 0.14 mm. In still further aspects, the first thickness can have any value that falls between any two foregoing values or it can fall within a range formed by any two foregoing values. For example, and without limitations, the first thickness can be about 0.06 m to about 0.14 mm, about 0.06 mm to about 0.13 mm, about 0.06 mm to about 0.12 mm, about 0.06 mm to about 0.11 mm, about 0.06 mm to about 0.10 mm, about 0.06 mm to about 0.09 mm, about 0.06 mm to about 0.08 mm, about 0.08 mm to about 0.15 mm, about 0.10 mm to about 0.15 mm, about 0.12 mm to about 0.15 mm, and so on.

In some aspects, this first thickness can be the same along the length l₁. While in other aspects, the first thickness can be uniform along some of the portions of the tubular member while gradually changing along other portions of the tubular member. For example, and without limitations, the thickness along l₂ can be the same but different from the thickness along l₃. In further exemplary aspects, the thickness along l₃ can be uniform, or it can also gradually change along l₃. In still further aspects, the thickness can change along l₂ and/or l₃ in any desired pattern. It is also understood that the thickness can vary both circumferentially and along a longitudinal axis. For example, it is understood that the first thickness can be but does not have to be uniform along a circumference of the tubular member. In still further aspects, the first thickness (or any thickness disclosed herein) can be thicker at the article surfaces that abut native anatomy when compared with the article surfaces that are further away from the native anatomy.

In some aspects, l₂ is greater than or equal to about 10 mm to about 16 mm, including exemplary values of about 10.5 mm, about 11 mm, about 11.5 mm, about 12 mm, about 12.5 mm, about 13 mm, about 13.5 mm, about 14 mm, about 14.5 mm, about 15 mm, and about 15.5 mm. In still further aspects, l₃ is about 2.5 mm to about 6 mm, including exemplary values of about 3 mm, about 3.5 mm, about 4 mm, about 4.5 mm, about 5 mm, and about 5.5 mm. In still further aspects, l₂ can have any value that falls between any two foregoing values, or it can fall within a range formed by any two foregoing values. For example, and without limitations, the l₂ can be about 10 mm to about 15 mm, about 10 mm to about 14 mm, about 10 mm to about 13 mm, about 10 mm to about 12 mm, about 10 mm to about 11 mm, about 11 mm to about 16 mm, about 12 mm to about 16 mm, about 14 mm to about 16 mm, and so on.

In still further aspects, the diameter d₁ of the tubular member can be the same or different along the length l₁. In still further aspects, the d₁ can be the same along l₂, as, for example, shown in FIG. 3. While in still further aspects, the d₁ can gradually change along l₂ (not shown). In still further aspects, the diameter d₁ can change to d₁′, d₁″, and/or d₁′″ along l₂. It is understood that in some aspects, d₁′ is the same as d₁″, and/or d₁′″. While in other aspects, d₁′, d₁″, and/or d₁′″ are all different depending on the desired application.

In still further aspects, d₁′, d₁″, and/or d₁′″ can have a value of about 2 mm to about 4 mm, including exemplary values of about 2.1 mm, about 2.2 mm, about 2.3 mm, about 2.4 mm, about 2.5 mm, about 2.6 mm, about 2.7 mm, about 2.8 mm, about 2.9 mm, about 3 mm, about 3.1 mm, about 3.2 mm, about 3.3 mm, about 3.4 mm, about 3.5 mm, about 3.6 mm, about 3.7 mm, about 3.8 mm, and about 3.9 mm. In still further aspects, the d₁′, d₁″, and/or d₁′″ can have any value that falls between any two foregoing values, or it can fall within a range formed by any two foregoing values. For example, and without limitations, the d₁′, d₁″, and/or d₁′″ can be about 2 mm to about 3.7 mm, about 2 mm to about 3.5 mm, about 2 mm to about 3.3 mm, about 2 mm to about 3 mm, about 2 mm to about 2.7 mm, about 2 mm to about 2.5 mm, about 2.2 mm to about 4 mm, about 2.5 mm to about 4 mm, about 2.7 mm to about 4 mm, about 3 mm to about 4 mm, about 3. 2 mm to about 4 mm, about 3.5 mm to about 4 mm, and so on.

In still further aspects, the woven article can further comprise a woven seal 3012 positioned at a distal end 3019 of the cushioning member 3007.

In still further aspects, each member of the disclosed article can have one or more layers if desired.

In still further aspects, the cushioning member 3007 can have any desired shape. In some aspects, the cushioning member can have a regular or irregular shape. For example, the cushioning member can have a spherical, cube, cuboid, pyramidal, oval, cone, or prism shape. In still further aspects, the cushioning member 3007 can have a regular or irregular cross-sectional shape. The cross-sectional shape of the cushioning member can be circular, oval, elliptical, rectangular, square, cross-sectional shape, or any combination thereof. In certain aspects, the cushioning member is symmetric. While in other aspects, the cushioning member is asymmetric. It is further understood that since the cushioning member is made of textile material, its shape is not rigid and can be deviated from the original shape upon use.

In still further aspects, the cross-sectional shape is defined by a length a₁ along at least one of the x- or y-axis. For example, as shown in FIG. 3, the cushioning member can have a₁ along the x-axis. In such exemplary and unlimiting aspects, length a₁ is the largest length that defines the cross-sectional shape. It is understood that in some aspects, the length along the y-axis can also be a₁. While in other aspects, the length along the y-axis can be a₁′, which is different from a₁. It is further understood that in such aspects, a₁ is greater than a₁′. It is understood, however, that in other exemplary aspects, a₁ can be along the y-axis, and it can be greater than a₁′ along the x-axis.

In still further aspects, a₁ can be about 3 mm to about 5 mm, including exemplary values of about 3.1 mm, about 3.2 mm, about 3.3 mm, about 3.4 mm, about 3.5 mm, about 3.6 mm, about 3.7 mm, about 3.8 mm, about 3.9 mm, about 4 mm, about 4.1 mm, about 4.2 mm, about 4.3 mm, about 4.4 mm, about 4.5 mm, about 4.6 mm, about 4.7 mm, about 4.8 mm, or about 4.9 mm. In still further aspects, the a₁ can have any value that falls between any two foregoing values, or it can fall within a range formed by any two foregoing values. For example, and without limitations, the a₁ can be about 3 mm to about 4.7 mm, about 3 mm to about 4.5 mm, about 3 mm to about 4.2 mm, about 3 mm to about 4 mm, about 3 mm to about 3.7 mm, about 3 mm to about 3.5 mm, about 3 mm to about 3.2 mm, about 3.2 mm to about 5 mm, about 3.5 mm to about 5 mm, about 3.7 mm to about 5 mm, about 4 mm to about 5 mm, about 4.2 mm to about 5 mm, about 4.5 mm to about 5 mm, and so on.

In still further aspects, the woven article described herein comprises a plurality of warp yarns and weft yarns.

In still further aspects, the plurality of warp yarns and the plurality of weft yarns comprise a biocompatible material. In some aspects, the plurality of warp yarns are the same or different from the plurality of weft yarns.

In some aspects, at least one of the plurality of warp yarns and/or a plurality of weft yarns can comprise one or more biocompatible textured multifilament yarn, a biocompatible textured high shrinkage multifilament yarn, a biocompatible flat multifilament yarn, a twisted multifilament yarn, or any combination thereof.

In still further aspects, the plurality of warp yarn and the plurality of weft yarns can comprise a textured yarn. It is understood that the textured yarn can be prepared by any known in the art methods. In certain aspects, the textured yarn can be selected from a friction-textured yarn, a false-twist textured yarn, a pin-textured yarn, an air-textured yarn, a belt-textured yarn, a stuffer box-textured yarn, or any combination thereof. Still, in further exemplary and unlimiting aspects, the textured yarn can be produced by friction texturing, or pin or false twist texturing, air texturing, stuffer box texturing, or any combination thereof. In still further exemplary aspects, the textured yarn is a friction-textured yarn.

In still further aspects, the yarns used in the disclosed article can be configured to shrink when treated. In such aspects, the yarn can exhibit a heat-shrinkage rate from about 10% to about 60%, including exemplary values of about 15%, about 20%, about 25%, about 30%, about 35%, about 40%, about 45%, about 40%, about 45%, about 50%, and about 55%. In still further aspects, the yarn can exhibit a heat-shrinkage rate having any value that falls between any two foregoing values or falling within a range formed by any two foregoing values. For example, and without limitations, the yarn can exhibit a heat-shrinkage rate of about 10% to about 55%, about 10% to about 50%, about 10% to about 45%, about 10% to about 40%, about 10% to about 35%, about 10% to about 30%, about 10% to about 20%, about 20% to about 60%, about 30% to about 60%, about 40% to about 60%, about 50% to about 60%, and so on.

It is further understood that various textured yarns can have various degrees of texturizing. The degree of texturing is evaluated by measuring crimp contraction, stretch potential, and the bulk ratio of the textured yarn. Without wishing to be bound by any theory, it is hypothesized that the degree of texturizing can have an impact on the shrinkage properties of the article as well as the final bulkiness of the various members of the article.

In some aspects and as disclosed herein, each yarn of the plurality of weft yarns and/or warp yarns can comprise a plurality of filaments. It is understood that the plurality of yarns present in the article can comprise the same or different yarns. For example, in certain aspects, and without limitation, all yarns in the plurality of yarns are textured. In still further aspects, all textured yarns are the same. However, in other aspects, not all the yarns comprise the same textured yarn. In certain aspects, the yarns can be different by a type of material or a type of texturing method. Similarly, the plurality of yarns in the weft and warp direction can be the same or different. It is understood that the plurality of weft and warp yarns can have various combinations of filaments and filament quantities. Still, further aspects, various yarn quantities, yarn texture types, and yarn texture degrees can also be utilized.

In still further aspects, each yarn of the plurality of weft yarns and/or textured warp yarns can have a size from about 10 to about 200 denier, including exemplary values of about 20 denier, about 50 denier, about 70 denier, about 100 denier, about 120 denier, about 150 denier, and about 170 denier. It is understood that each yarn of the plurality of weft yarns and/or warp yarns can have a denier value between any two foregoing values. For example, and without limitation, each yarn of the plurality of weft yarns and/or warp yarns can have a denier value from about 12 denier to about 25 denier, or from about 30 denier to about 85 denier, or from about 110 denier to about 185 denier.

In still further aspects, each yarn of the plurality of weft yarns and/or warp textured yarns can comprise from about 8 to about 150 filaments, including exemplary values of about 10 filaments, about 15 filaments, about 20 filaments, about 50 filaments, about 70 filaments, about 100 filaments, and about 120 filaments. It is understood that each yarn of the plurality of weft yarns and/or warp yarns can comprise any number of fibers between any two foregoing values. For example, and without limitation, each yarn of the plurality of weft yarns and/or warp yarns can comprise from about 8 to 50 filaments, from about 25 filaments to about 85 filaments, or from about 110 filaments to about 145 filaments.

In still further aspects, the filaments can have any diameter suitable for the desired application. In certain aspects, the filaments can have a diameter from about 1 μm to about 25 μm, including exemplary values of about 2 μm, about 5 μm, about 7 μm, about 10 μm, about 12 μm, about 15 μm, about 17 μm, about 20 μm, and about 22 μm. It is understood that the filaments can have any diameter between any two foregoing values. For example, and without limitation, the filaments can have a diameter from about 3 μm to about 8 μm, or from about 11 μm to about 22 μm, or from about 15 μm to about 25 μm.

In still further aspects, the plurality of warp yarns and a plurality of weft yarns comprise polyesters, aliphatic polyesters, co-polyesters, polyolefins, fluoropolymers, polyurethanes, polyethers, polyureas, nylons, natural fibers, copolymers thereof, or a combination thereof.

In still further aspects, the plurality of warp yarns and a plurality of weft yarns can comprise ultra-high molecular weight polyethylene, polyethylene, polypropylene, polytetrafluoroethylene, expanded polytetrafluorethylene, polyvinylidene fluoride, or any combination thereof.

In still further aspects, the plurality of warp yarns and a plurality of weft yarns comprise non-resorbable yarns, bio-resorbable yarns, or a combination thereof. For example, and without limitations, the bio-resorbable yarns comprise one or more aliphatic polyester or co-polyesters, or a combination thereof. In still further aspects, the bio-resorbable yarns can comprise polyglycolic acid (PGA), poly(L-lactic acid) (PLLA), polycaprolactone (PCL), polylactide glycolide copolymers (PLGA), copolymers thereof, or a combination thereof.

In certain aspects, such a yarn can also comprise composite filaments. It is understood that, as used herein, the composite filaments relate to a filament that can comprise one or more different materials. In certain aspects, the composite filament is a bicomponent filament that can have any configuration, for example, and without limitation, a side-by-side configuration, a core-sheath configuration, a segmented configuration, an islands-in-the-sea configuration, or any combination thereof.

The tubular member of the woven article disclosed herein can have any weave pattern suitable for the desired application. For example, and without limitations, the tubular member can have a plain weave pattern, a twill weave, a satin weave, any derivative, or any combination thereof.

In some aspects, the cushioning member has a 3-D honeycomb pattern having a plurality of repeating cell units. It is understood that also enclosed are aspects wherein a portion of the cushioning member has the 3-D honeycomb pattern. While in other aspects, an entire cushioning member has a 3-D honeycomb pattern.

In still further aspects, the plurality of repeating cell units can comprise about 1 to about 32 cells per inch, including exemplary values of about 2, about 5, about 8, about 10, about 12, about 15, about 18, about 20, about 22, about 25, about 28, and about 30 cells per inch. In still further aspects, the plurality of repeating cell units can have any value that falls between any two foregoing values or can fall within a range formed by any two foregoing values. For example, and without limitations, the plurality of repeating cell units can be about 1 to about 28 cells per inch, about 1 to about 24 cells per inch, about 1 to about 20 cells per inch, about 1 to about 16 cells per inch, about 1 to about 8 cells per inch, about 8 to about 32 cells per inch, about 16 to about 32 cells per inch, about 20 to about 32 cells per inch, about 24 to about 32 cells per inch, and so on.

In still further aspects, each of the plurality of repeating cell units comprises about 8 to about 50 weft yarn insertion repeats per linear inch, including exemplary values of about 10, about 12, about 15, about 18, about 20, about 22, about 25, about 28, about 30, about 32, about 35, about 38, about 40, about 42, about 45, and about 48 weft yarn insertion repeats per linear inch. In still further aspects, the weft yarn insertion repeats per linear inch can have any value that falls between any two foregoing values or can fall within a range formed by any two foregoing values. For example, and without limitations, the weft yarn insertion repeats per linear inch can be about 8 to about 45, about 8 to about 40, about 8 to about 30, about 8 to about 20, about 10 to about 50, about 16 to about 50, about 24 to about 50, and so on.

Exemplary honeycomb patterns are described, for example, in WO2022/226090 and WO2021/188405, the contents of which are incorporated herein by reference for all purposes.

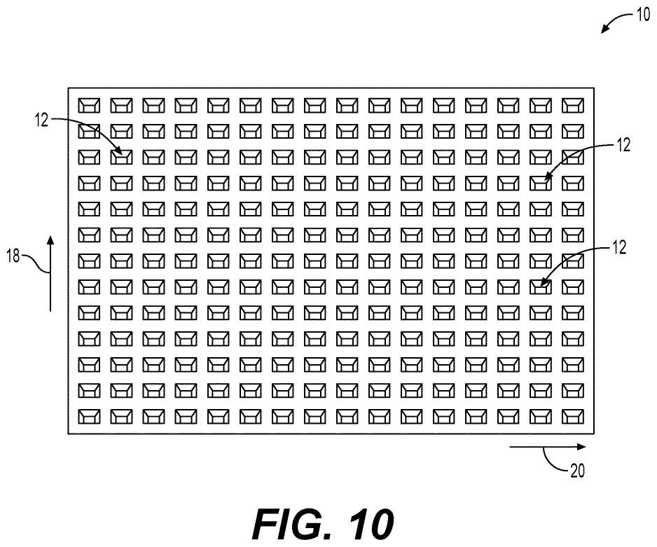

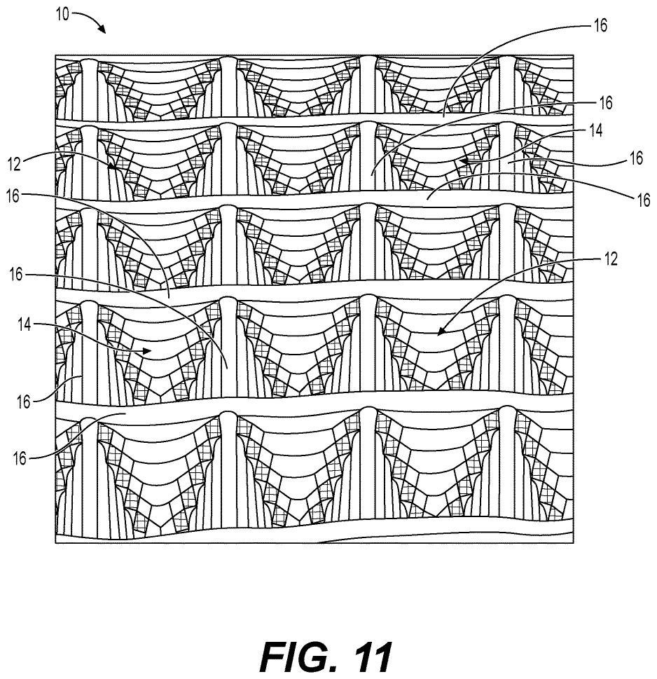

An exemplary and unlimiting aspect of the honeycomb pattern for a planar representation 10 of the cushioning material is shown in FIG. 10. In this example, the honeycomb weave pattern can comprise a repeating pattern of cells 12. Cells 12 can have portions with varied heights, with a central portion 14 (represented in FIG. 11) having a lower height than an outer portion 16 (represented in FIG. 11). The central portion 14 can form a pit that is surrounded by the outer portion 16. The outer portion 16 can comprise raised walls that surround the pit of the central portion 14. Each cell 12 can include a pit surrounded by walls. In some aspects, the pits can have a variety of shapes, including an inverted pyramidal shape, a domed shape, or another shape as desired. The cells 12 can have a square or rectangular shape, as shown in FIGS. 10-11 or can have another shape as desired.

The cells 12 can repeat along a length 18 and a width 20 of the planar representation 10 of the cushioning material. It is understood that since the cushioning material described herein has a 3-D shape, the cells 12 can repeat along all perimeter of the cushioning material. The cells 12 can be adjacent to each other, with the outer portion 16 of each cell 12 being adjacent to an outer portion 16 of an adjacent cell, as shown in FIG. 11, for example. As shown in FIGS. 10 and 11, the cells can be aligned with each other along the length 18 (in columns) and the width 20 (in rows) in examples, or other patterns can be utilized as desired. For example, the cell 12 can be aligned in a diagonal direction or another pattern in examples as desired. The cells 12 can repeat with irregular configurations; for example, one cell 12 can have a square configuration, as shown in FIG. 11, for example, and an adjacent cell can have another configuration in examples.

In still further aspects, the honeycomb weave pattern can be formed by warp and weft threads interlaced and floating in a way that creates the outer portions 16 (e.g., the walls) and the central portion 14 (e.g., the pits) in a regular pattern. In some aspects, the warp and weft floats can be arranged around a plain weave center and can be woven partly on tabby areas surrounded by ridges of long floats. Other configurations can be utilized as desired.

It is understood, however, that other configurations of weaves can also be utilized. For example, between a four-end honeycomb repeat to a thirty-two-end honeycomb can be utilized in some aspects. A greater configuration than a thirty-two-end honeycomb repeat can be utilized in some aspects. A range between a four-end honeycomb repeat and a sixteen-end honeycomb repeat can be utilized in some aspects. A range between a sixteen-end honeycomb repeat and a thirty-two-end honeycomb repeat can be utilized in some aspects. Other configurations can be utilized as desired.

In some aspects, the honeycomb weave pattern can include honeycomb weave derivatives such as a modified honeycomb weave pattern or other derivatives as desired.

FIG. 11 illustrates a perspective view of a representation of a honeycomb weave pattern according to aspects disclosed herein. In some aspects, certain details of the weave pattern can be excluded from view.

In certain aspects, the repeating pattern of cell 12 is visible, including the pits of the central portion 14 surrounded by the walls of the outer portion 16 for each cell 12. A variation in height between the pits of the central portion 14 and the walls of the outer portion 16 is visible.

In still further aspects, the cushioning member can have a second thickness. The second thickness can be about 1.5 mm to about 5 mm, including exemplary values of about 1.8 mm, about 2 mm, about 2.2 mm, about 2.5 mm, about 2.8 mm, about 3 mm, about 3.2 mm, about 3.5 mm, about 3.8 mm, about 4 mm, about 4.2 mm, about 4.5 mm, and about 4.8 mm. In still further aspects, the second thickness can have any value that falls between any two foregoing values or can fall within a range formed by any two foregoing values. For example, and without limitations, the second thickness can be about 1.5 mm to about 4.5 mm, 1.5 mm to about 4 mm, 1.5 mm to about 3.5 mm, 1.5 mm to about 3 mm, 1.5 mm to about 2.5 mm, 1.5 mm to about 2 mm, 2 mm to about 4.5 mm, 2.5 mm to about 4.5 mm, 3 mm to about 4.5 mm, 3.5 mm to about 4.5 mm, and so on.

In still further aspects, the first thickness of the tubular member is smaller than the second thickness of the cushioning member. In still further aspects, the second thickness is the same or different along a circumference and/or a perimeter of the cushioning member and/or along a length of the cushioning member.

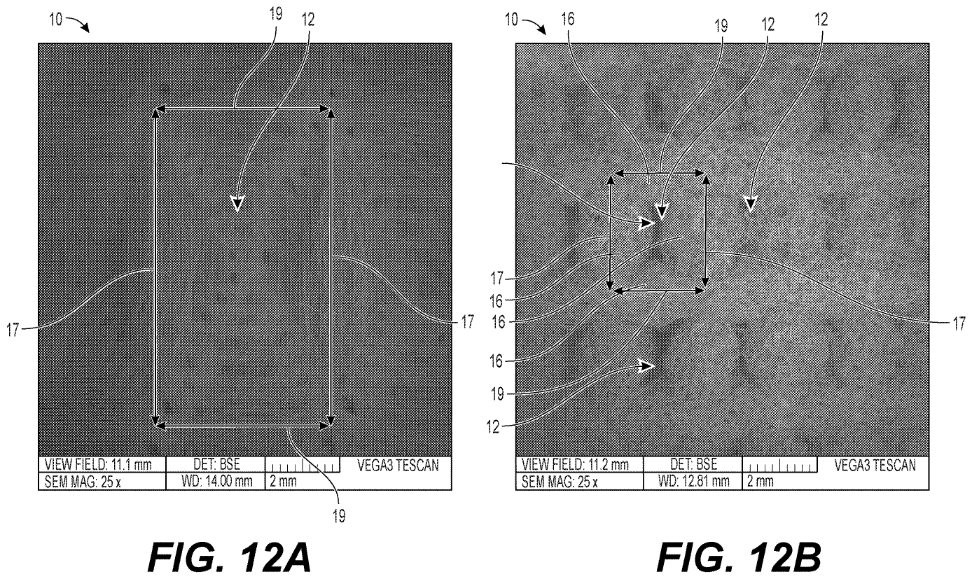

As disclosed above, the yarn used to construct the disclosed herein article can exhibit a heat shrinkage when heated at a desired temperature for a desired time. An example of such heat shrinkage is shown in FIGS. 12A-12B. FIG. 12A, for example, illustrates a portion of a surface of the cushioning member 10 prior to heat treatment. A length 17 and width 19 of an exemplary cell unit 12 are marked. FIG. 12B illustrates the portion of a surface of the cushioning member 10 shown in FIG. 12A following heat treatment, with the length 17 and width 19 of the cell unit 12 having decreased, while the second thickness of the cushioning member has increased.

In still further aspects, the cushioning gradient is achieved by varying the weave density, varying the quantity of repeating cell units, varying a number of the weft yarn insertions in the plurality of weft yarns, varying the denier of the weft yarns, varying the texture of the weft yarns, or by a combination thereof. The cushioning gradient can be achieved by varying the number of repeating cell units within the cushioning member. For example, and as discussed above, the repeating cell units can be varied between about 1 and about 32 cells per inch, including repeating units having values from about 2, about 5, about 8, about 10, about 12, about 15, about 18, about 20, about 22, about 25, about 28, and about 30 cells per inch. The number of cells per inch can fall between any two foregoing values or within any range formed by any two foregoing values. For example, and without limitations, about 1 and about 30 cells per inch, about 1 and about 28 cells per inch, about 1 and about 24 cells per inch, about 1 and about 20 cells per inch, about 1 and about 16 cells per inch, about 1 and about 10 cells per inch, about 5 and about 32 cells per inch, about 8 and about 32 cells per inch, about 10 and about 32 cells per inch, about 12 and about 32 cells per inch, about 16 and about 32 cells per inch, and so on.

The cushioning gradient can be further achieved within the cushioning member by varying weft yarn insertions as discussed above, from about 8 to about 50 per linear inch, including exemplary values of about 10, about 12, about 15, about 18, about 20, about 22, about 25, about 28, about 30, about 32, about 35, about 38, about 40, about 42, about 45, and about 48 weft yarn insertion repeats per linear inch. It is understood that the cushioning member can have any number of weft yarn insertion repeats per linear inch that falls between any two foregoing values or within the range formed by any two foregoing values.

The cushioning gradient can also be achieved within the cushioning member by varying the weft yarn denier from about 10 to about 200 denier, including exemplary values of about 20 denier, about 50 denier, about 70 denier, about 100 denier, about 120 denier, about 150 denier, and about 170 denier. It is understood that a weft yarn denier can have any value that falls between any two foregoing values or within a range formed by any two foregoing values. For example, the denier of the weft yarn can be about 10 to about 150 denier, about 10 to about 100 denier, about 10 to about 70 denier, about 10 to about 50 denier, about 50 to about 200 denier, about 70 to about 200 denier, about 100 to about 200 denier, about 150 to about 200 denier, and so on. Even further, the cushioning gradient can be achieved by varying the weft yarns from not textured (flat or FDY yarns) to highly textured yarns.

Referring back to FIG. 3, it can be seen that the woven article, as disclosed herein, can transition into the cushioning member 3007 at the proximal end 3009 of the cushioning member such that a first tapered-out portion 3015 of the cushioning member 3007 is formed. In such exemplary and unlimiting aspects, the first tapered-out portion 3015 can be formed by increasing the spacing between a plurality of warp yarns during a weft yarn insertion in a weaving process. Yet in other exemplary and unlimiting aspects, the first tapered-out portion 3015 is formed by adding at least one warp yarn during a weaving process. In some aspects, the tapering-out portion can be measured by an offset from an imaginary line 3300 that extends from the outer surface of the tubular member along a longitudinal axis z of the article. In such aspects, a length 3320 measured at the middle of the offset can be about 0.5 mm to about 1.5 mm, including exemplary values of about 0.6 mm, about 0.7 mm, about 0.8 mm, about 0.9 mm, about 1 mm, about 1.1 mm, about 1.2 mm, about 1.3 mm, and about 1.4 mm. The length can also have any value falling between any two foregoing values or within a range formed by any two foregoing values. For example, the length can be about 0.5 mm to about 1.2 mm, about 0.5 mm to about 1 mm, about 0.5 mm to about 0.8 mm, about 0.8 mm to about 1.5 mm, about 1 mm to about 1.5 mm, about 1.2 mm to about 1.5 mm, and so on.

In still further aspects, the first tapered-out portion 3015 can have a length 3006 of about 2 mm to about 3 mm, including exemplary values of about 2.1 mm, about 2.2 mm, about 2.3 mm, about 2.4 mm, about 2.5 mm, about 2.6 mm, about 2.7 mm, about 2.8 mm, and about 2.9 mm. The length can also have any value falling between any two foregoing values or within a range formed by any two foregoing values. For example, the length can be about 2 mm to about 2.9 mm, about 2 mm to about 2.7 mm, about 2 mm to about 2.5 mm, about 2 mm to about 2.2 mm, about 2.2 mm to about 3 mm, about 2.5 mm to about 3 mm, about 2.7 mm to about 3 mm, and so on.

Still, further, the distal portion of the cushioning member 3007 can comprise a first tapered-in portion 3017. In such aspects, the first tapered-in portion 3017 terminates in the distal end 3019 of the cushioning member 3007, wherein the distal end of the cushioning member transitions into the woven seal 3012. In yet still further aspects, the first tapered-in portion 3017 is formed by decreasing a spacing between a plurality of warp yarns during a weft yarn insertion in a weaving process. In still further aspects, the first tapered-in portion 3017 is formed by removing at least one warp yarn during a weaving process. In yet still further aspects, the first tapered-in portion 3017 is terminated by the woven seal 3012.

In some aspects, the first tapering-in portion can be measured by an offset from an imaginary line 3300 that extends from the outer surface of the tubular member along a longitudinal axis z of the article. In such aspects, a length 3340 measured at the middle of the offset can be about 0.5 mm to about 1.5 mm, including exemplary values of about 0.6 mm, about 0.7 mm, about 0.8 mm, about 0.9 mm, about 1 mm, about 1.1 mm, about 1.2 mm, about 1.3 mm, and about 1.4 mm. The length can also have any value falling between any two foregoing values or within a range formed by any two foregoing values. For example, the length can be about 0.5 mm to about 1.2 mm, about 0.5 mm to about 1 mm, about 0.5 mm to about 0.8 mm, about 0.8 mm to about 1.5 mm, about 1 mm to about 1.5 mm, about 1.2 mm to about 1.5 mm, and so on.

In still further aspects, the first tapered-in portion 3017 can have a length 3008 of about 2 mm to about 3 mm, including exemplary values of about 2.1 mm, about 2.2 mm, about 2.3 mm, about 2.4 mm, about 2.5 mm, about 2.6 mm, about 2.7 mm, about 2.8 mm, and about 2.9 mm. The length can also have any value falling between any two foregoing values or within a range formed by any two foregoing values. For example, the length can be about 2 mm to about 2.9 mm, about 2 mm to about 2.7 mm, about 2 mm to about 2.5 mm, about 2 mm to about 2.2 mm, about 2.2 mm to about 3 mm, about 2.5 mm to about 3 mm, about 2.7 mm to about 3 mm, and so on.

It is understood that the length of the offset of the first tapered-out portion and the length of the offset of the first tapered-in portion can be the same or different depending on the desired shape.

In still further aspects, a portion of the cushioning member disposed between the first tapered-out and first tapered-in portions can have a length 3010 from about 2 mm to about 3 mm, including exemplary values of about 2.1 mm, about 2.2 mm, about 2.3 mm, about 2.4 mm, about 2.5 mm, about 2.6 mm, about 2.7 mm, about 2.8 mm, and about 2.9 mm. The length can also have any value falling between any two foregoing values or within a range formed by any two foregoing values. For example, the length can be about 2 mm to about 2.9 mm, about 2 mm to about 2.7 mm, about 2 mm to about 2.5 mm, about 2 mm to about 2.2 mm, about 2.2 mm to about 3 mm, about 2.5 mm to about 3 mm, about 2.7 mm to about 3 mm, and so on.

In some aspects, the woven seal 3012 has a length 3013 in a longitudinal direction of about 1 mm to about 3 mm, including exemplary values of about 1.2 mm, about 1.5 mm, about 1.8 mm, about 2 mm, about 1.2 mm, about 1.5 mm, and about 1.8 mm. The length can also have any value falling between any two foregoing values or within a range formed by any two foregoing values. For example, the length can be about 1 mm to about 2.9 mm, about 1 mm to about 2.7 mm, about 1 mm to about 2.5 mm, about 1 mm to about 2.2 mm, about 1 mm to about 2 mm, about 1 mm to about 1.8 mm, about 1 mm to about 1.5 mm, about 1.2 mm to about 3 mm, about 1.5 mm to about 3 mm, about 1.8 mm to about 3 mm, about 2 mm to about 3 mm, about 2.2 mm to about 3 mm, about 2.5 mm to about 3 mm, about 2.7 mm to about 3 mm, and so on.

In still further aspects, the woven seal 3012 can have a length along of x-axis from about 2 mm to about 3 mm, including exemplary values of about 2.1 mm, about 2.2 mm, about 2.3 mm, about 2.4 mm, about 2.5 mm, about 2.6 mm, about 2.7 mm, about 2.8 mm, and about 2.9 mm. The length can also have any value falling between any two foregoing values or within a range formed by any two foregoing values. For example, the length can be about 2 mm to about 2.9 mm, about 2 mm to about 2.7 mm, about 2 mm to about 2.5 mm, about 2 mm to about 2.2 mm, about 2.2 mm to about 3 mm, about 2.5 mm to about 3 mm, about 2.7 mm to about 3 mm, and so on.

In still further aspects, the second end 3005 of the tubular member 3002 positioned within the central cavity 3011 of the cushioning member is closed. In some aspects, the portion 3004 of the lumen positioned within the central cavity 3011 of the cushioning member 3007 behaves as an inner pocket of the article.

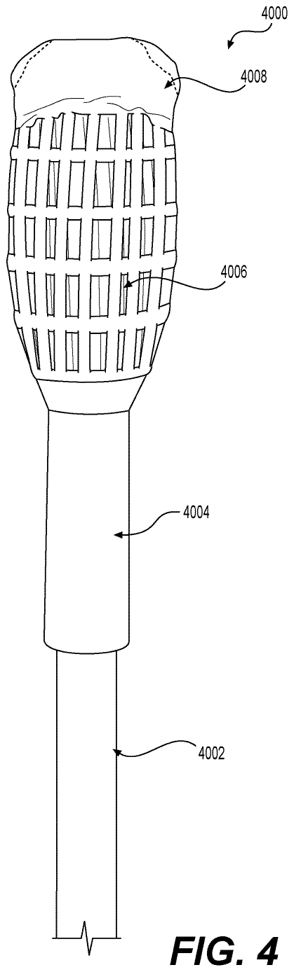

FIG. 4 shows an exemplary woven article 4000 disposed on a mandrel 4002, having a tubular member 4004, a cushioning member 4006, and a woven seal 4008. The portion of the tubular member that forms an inner pocket, as disclosed above, is not shown.

In still further aspects, a length of the article from an open proximal end of the tubular member up to a woven seal can be about 17 mm to about 20 mm, including exemplary values of about 17.2 mm, about 17.5 mm, about 17.8 mm, about 18 mm, about 18.2 mm, about 18.5 mm, about 18.8 mm, about 19 mm, about 19.2 mm, about 19.5 mm, and about 19.8 mm. The length can also have any value falling between any two foregoing values or within a range formed by any two foregoing values. For example, the length can be about 17 mm to about 19.5 mm, about 17 mm to about 19 mm, about 17 mm to about 18.5 mm, about 17 mm to about 18 mm, about 17.5 mm to about 20 mm, about 18.5 mm to about 20 mm, about 19 mm to about 20 mm, and so on.

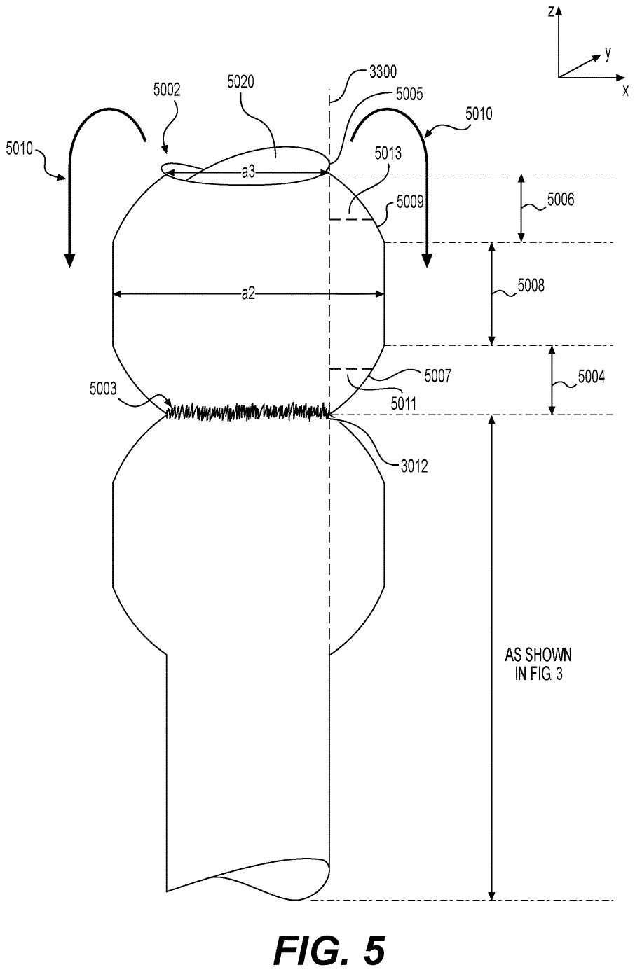

In still further aspects, the woven article can further comprise a covering member 5002, as shown in FIG. 5. In such aspects, the covering member can have a third thickness of about 0.06 mm to about 0.15 mm, including exemplary values of about 0.07 mm, about 0.08 mm, about 0.09 mm, about 0.1 mm, about 0.11 mm, about 0.12 mm, about 0.13 mm, and about 0.14 mm. The thickness can also have any value falling between any two foregoing values or within a range formed by any two foregoing values. For example, the thickness can be about 0.06 mm to about 0.12 mm, about 0.06 mm to about 0.1 mm, about 0.06 mm to about 0.08 mm, about 0.08 mm to about 0.15 mm, about 0.1 mm to about 0.15 mm, about 0.12 mm to about 0.15 mm, and so on. In still further aspects, the third thickness is the same or different along a circumference and/or perimeter of the covering member and/or along a length of the covering member.

In some aspects, the covering member is defined by an inner surface and an outer surface. In some aspects, the covering member 5002 has a proximal end 5003 and a distal end 5005, wherein the proximal end 5003 of the covering member is coupled to the woven seal 3012, similar to the one described in FIG. 3. In still further aspects, the distal end 5005 is open.

In still further aspects, the coupling between the woven seal 3012 and the proximal end 5003 of the covering member is formed during a weaving process, thus forming a unibody article.

In still further aspects, the covering member 5002 can have any desired shape. In some aspects, the covering member can have a regular or irregular shape. For example, the covering member can have a spherical, cube, cuboid, pyramidal, oval, cone, or prism shape. In still further aspects, the covering member 5002 can have a regular or irregular cross-sectional shape. The cross-sectional shape of the covering member can be circular, oval, elliptical, rectangular, square, cross-sectional shape, or any combination thereof. In certain aspects, the covering member is symmetric. While in other aspects, the covering member is asymmetric. It is further understood that since the covering member is made of textile material, its shape is not rigid and can be deviated from the original shape upon use.

In still further aspects, the cross-sectional shape of the covering member is defined by a length a₂ along at least one of the x- or y-axis. For example, as shown in FIG. 5, the covering member can have length a₂ along the x-axis. In such exemplary and unlimiting aspects, length a₂ is the largest length that defines the cross-sectional shape. It is understood that in some aspects, the length along the y-axis can also be a₂. While in other aspects, the length along the y-axis can be a₂′, which is different from a₂. It is further understood that in such aspects, a₂ is greater than a₂′. It is understood, however, that in other exemplary aspects, a₂ can be along the y-axis, and it can be greater than a₁′ along the x-axis.

In still further aspects, the length a₂ along at least one of the x- or y-axis of the covering member is greater than the length a₁ of the cushioning member.

In still further aspects, the covering member has a plain weave pattern, a twill weave, a satin weave, any derivative, or any combination thereof weave patterns. In some aspects, the weave pattern of the covering member can be the same as the weave of the tubular member. Yet, in other aspects, the weave patterns of the covering member and the tubular member are different. In still further aspects, the covering member can have a density higher than the tubular member. While in other aspects, the covering member can have a density that is lower than the tubular member. Yet in still other aspects, the densities of the tubular member and covering member are the same.

In further aspects, the covering member comprises a second tapered-out portion 5007 positioned outwards and up from the proximal end 5003 of the covering member 5002. Yet in other exemplary and unlimiting aspects, the second tapered-out portion 5007 is formed by adding at least one warp yarn during a weaving process. In some aspects, the tapering-out portion 5007 can be measured by an offset from an imaginary line 3300 that extends from the outer surface of the tubular member along a longitudinal axis z of the article. In such aspects, a length 5011 measured at the middle of the offset can be about 0.5 mm to about 1.5 mm, including exemplary values of about 0.6 mm, about 0.7 mm, about 0.8 mm, about 0.9 mm, about 1 mm, about 1.1 mm, about 1.2 mm, about 1.3 mm, and about 1.4 mm. In still further aspects, the second tapered-out portion 5007 can have a length 5004 of about 2 mm to about 3 mm, including exemplary values of about 2.1 mm, about 2.2 mm, about 2.3 mm, about 2.4 mm, about 2.5 mm, about 2.6 mm, about 2.7 mm, about 2.8 mm, and about 2.9 mm. The lengths 5011 and 5004 can have any value that falls between any two respective foregoing values or within the range formed by any two respective foregoing values.

In still further aspects, the covering member can comprise a second tapered-in portion 5009 at the distal portion of the covering member, wherein the second tapered-in portion terminates in the open distal end 5020 of the covering member having a length a₃ along at least one of x or y-axis. In still further aspects, the length a₃ can be smaller than a₂ and equal to or greater than the length a₁.

Still further, the distal portion of the covering member 5002 can comprise a second tapered-in portion 5009. In such aspects, the second tapered-in portion 5009 terminates in the open distal end 5020 of the covering member 5002. In yet still further aspects, the second tapered-in portion 5009 is formed by decreasing a spacing between a plurality of warp yarns during a weft yarn insertion in a weaving process. In still further aspects, the second tapered-in portion 5009 is formed by removing at least one warp yarn during a weaving process. The covering member also has a middle portion 5008 disposed between 5007 and 5009.

In some aspects, the second tapering-in portion can be measured by an offset from an imaginary line 3300 that extends from the outer surface of the tubular member along a longitudinal axis z of the article. In such aspects, a length 5013 measured at a middle of the offset can be about 0.5 mm to about 1.5 mm, including exemplary values of about 0.6 mm, about 0.7 mm, about 0.8 mm, about 0.9 mm, about 1 mm, about 1.1 mm, about 1.2 mm, about 1.3 mm, and about 1.4 mm. The length 5013 can also have any value falling between any two foregoing values or within a range formed by any two foregoing values. For example, the length can be about 0.5 mm to about 1.2 mm, about 0.5 mm to about 1 mm, about 0.5 mm to about 0.8 mm, about 0.8 mm to about 1.5 mm, about 1 mm to about 1.5 mm, about 1.2 mm to about 1.5 mm, and so on.

In still further aspects, the second tapered-in portion 5009 can have a length 5006 of about 2 mm to about 3 mm, including exemplary values of about 2.1 mm, about 2.2 mm, about 2.3 mm, about 2.4 mm, about 2.5 mm, about 2.6 mm, about 2.7 mm, about 2.8 mm, and about 2.9 mm. The length 5006 can also have any value falling between any two foregoing values or within a range formed by any two foregoing values. For example, the length can be about 2 mm to about 2.9 mm, about 2 mm to about 2.7 mm, about 2 mm to about 2.5 mm, about 2 mm to about 2.2 mm, about 2.2 mm to about 3 mm, about 2.5 mm to about 3 mm, about 2.7 mm to about 3 mm, and so on.

It is understood that the length of the offset of the second tapered-out portion and the length of the offset of the second tapered-in portion can be the same or different depending on the desired shape. It is also understood that the length of the offset of the first and second tapered-out portions and the length of the offset of the first and the second tapered-in portions can be the same or different depending on the desired shape.

In still further aspects, a portion of the covering member disposed between the second tapered-out and second tapered-in portions can have a length from about 2 mm to about 3 mm, including exemplary values of about 2.1 mm, about 2.2 mm, about 2.3 mm, about 2.4 mm, about 2.5 mm, about 2.6 mm, about 2.7 mm, about 2.8 mm, and about 2.9 mm. The length can also have any value falling between any two foregoing values or within a range formed by any two foregoing values. For example, the length can be about 2 mm to about 2.9 mm, about 2 mm to about 2.7 mm, about 2 mm to about 2.5 mm, about 2 mm to about 2.2 mm, about 2.2 mm to about 3 mm, about 2.5 mm to about 3 mm, about 2.7 mm to about 3 mm, and so on.

In still further aspects, the covering member is configured to be folded, as schematically shown by arrow 5010, over the cushioning member such that the outer surface of the covering member is positioned above the outer surface of the cushioning member and the inner surface of the covering member is exposed to a surrounding environment.

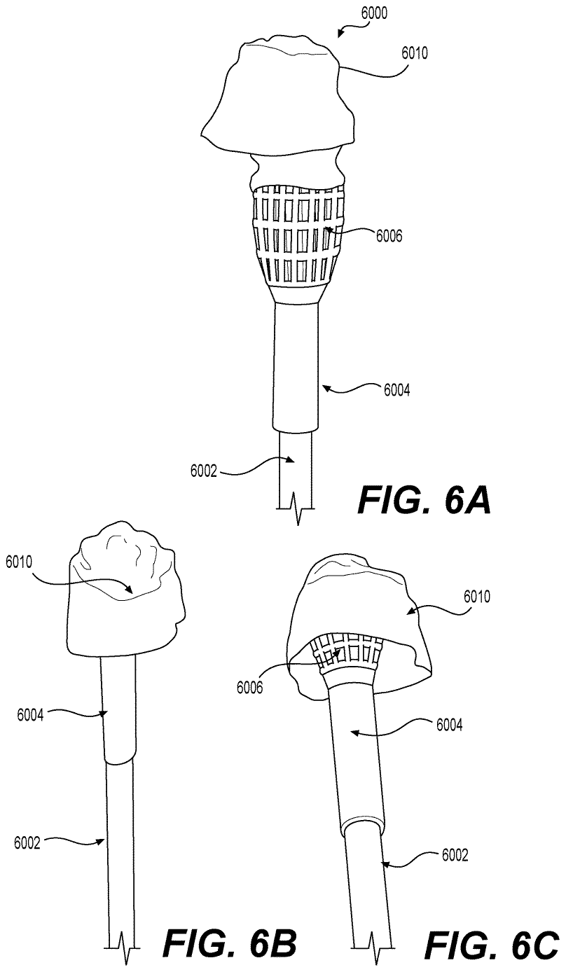

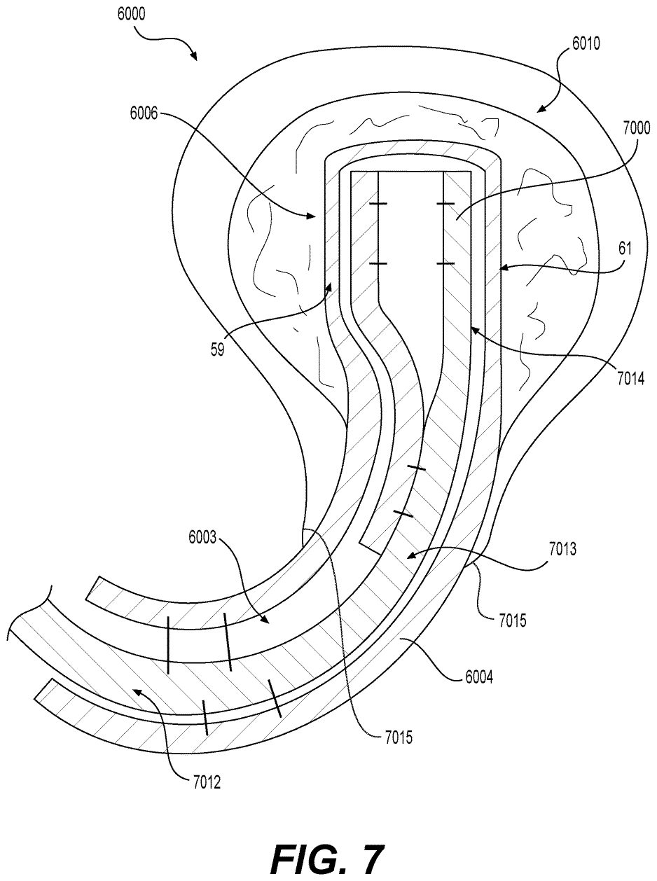

The exemplary articles comprising the covering members are shown in FIGS. 6A-6C. For example, and as seen in FIG. 6A, the woven article 6000 is positioned on a mandrel 6002, wherein the tubular member 6004 transitions into a cushioning member 6006, and the covering member 6010 starts to be folded over the cushioning member. In FIGS. 6B-6C, the cushioning member 6006 is below the covering member 6010.

In still further aspects, the woven article is configured to be sutured or attached to a desired substrate by any known in the art methods. In certain aspects, the woven article can comprise one or more apertures disposed circumferentially (or along a perimeter of the different members) and/or longitudinally in predetermined locations. In such aspects, the one or more apertures are configured to receive a fastening member for an attachment of the woven article to a substrate. In such aspects, the fastening member can be a suture. However, it can also be any other fastening member that is suitable for the desired application. It is understood that any known in the art sutures can be used. In certain exemplary and unlimiting aspects, the suture is a textile filament. In yet other aspects, the suture is any filament that can be used for the desired application.

In certain aspects, the woven article disclosed herein can be used in implantable medical devices. In aspects disclosed herein, the woven article described above can be configured to be assembled in an implantable medical device comprising: one or more anchors, wherein each of the one or more anchors is configured to anchor the implantable medical device into a patient's natural anatomy.

In still further aspects, the woven article is configured to receive an anchor of the one or more anchors through the first open end of the tubular member such that the anchor is positioned within the lumen of the tubular member, such that the second end of the tubular member substantially prevent a further anchor's movement within the article. In still further aspects, the cushioning member protects the patient's natural anatomy. While in still further aspects, the covering member, if present, is configured to form a substantially smooth surface that is exposed to the patient's natural anatomy.

In yet still further aspects, the woven article is configured to promote a tissue response. In still further aspects, the woven article can behave as a graft material.

### Implantable Medical Device

It is understood that any of the articles described above can be used in an implantable medical device.

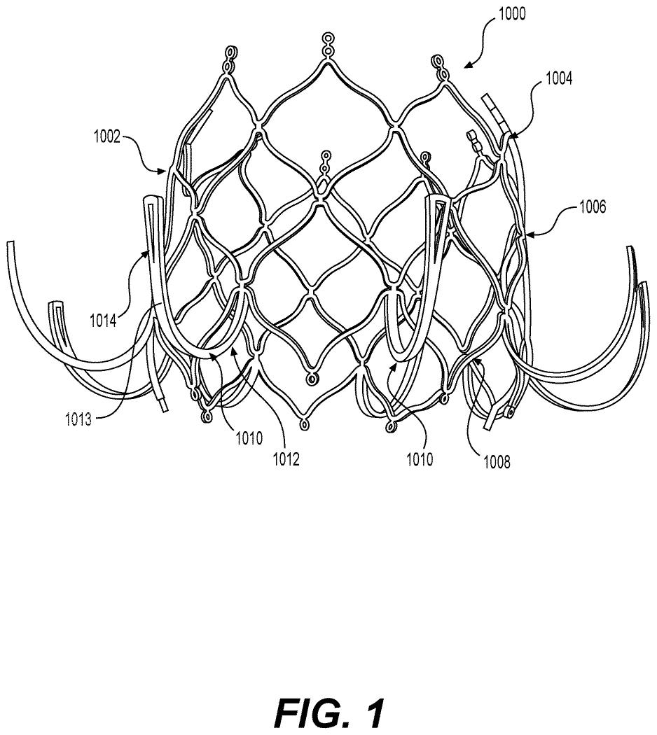

In certain aspects, the exemplary implantable medical device can comprise a frame 1000, as shown in FIG. 1. In such an aspect, the frame 1000 is shown in an expanded configuration. The frame 1000 can include a frame portion 1002 having an upper region 1004, an intermediate region 1006, and a lower region 1008. In certain aspects, the intermediate region 1006 can have a greater diameter than the diameter of the frame 1000 at the proximal (abut to the upper region) and/or distal (abut the lower region) ends when the frame 1000 is in an expanded configuration. In some aspects, such as the illustrated aspect, the frame 1000 can include an intermediate region 1006, which has a greater cross-sectional area than the cross-sectional area of the frame 1000 at the proximal (abut to the upper region) and/or distal (abut the lower region) ends when the frame 1000 is in an expanded configuration. A longitudinal axis (not shown) of the frame 1000 can be defined as the central axis that extends through the center of the frame 1000 between the upper and lower ends of the frame 1000. In some aspects, the frame 1000 can be oriented such that the upper region 1004 is a proximal portion, and the lower region 1008 is a distal portion. The frame 1000 can include a plurality of anchoring members 1010. In some aspects, the frame 1000 can be oriented such that the plurality of anchoring members 1010 are distal anchoring members. As shown in the illustrated aspect, the plurality of anchoring members 1010 can include one or more anchors. For example, and without limitation, as shown in the illustrated aspect, the plurality of anchoring members 1010 can include nine anchors, each including a neck 1012 and arm 1013 extending from a lower region 1008 of the frame portion 1002 and a tip 1014. It is understood, however, that the implantable device can have more than 9 anchors or less than 9 anchors. For example, the device can have 1, 2, 3, 4, 5, 6, 7, 8, 9, 10, 11, or 12 anchors. As shown in FIG. 1, in some aspects, each of the one or more anchors has a hook shape. In certain aspects, the one or more anchors comprise ventricular anchors.

In still further aspects, the one or more anchors of the medical device are positioned within the disclosed herein article.

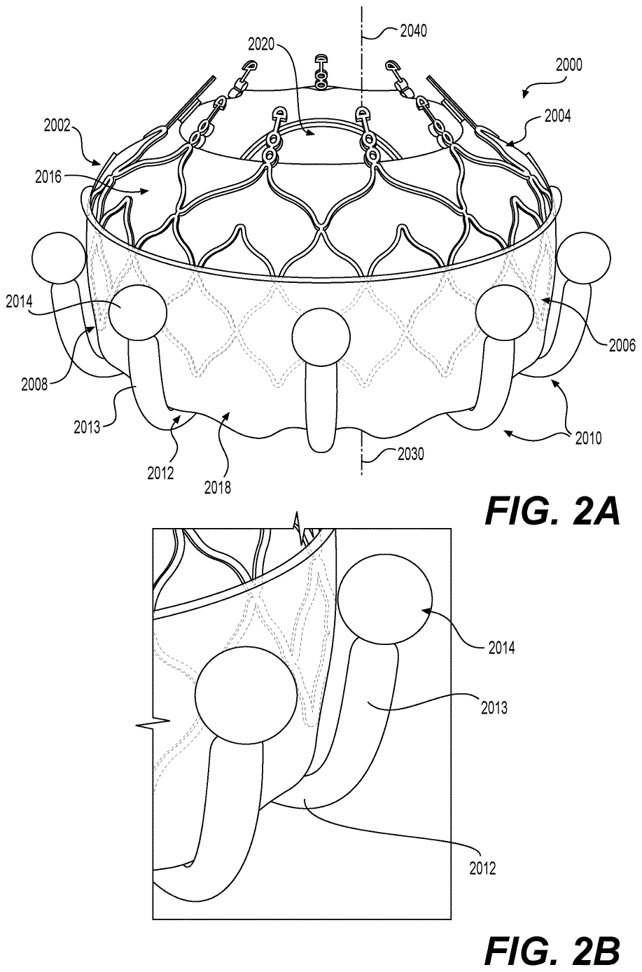

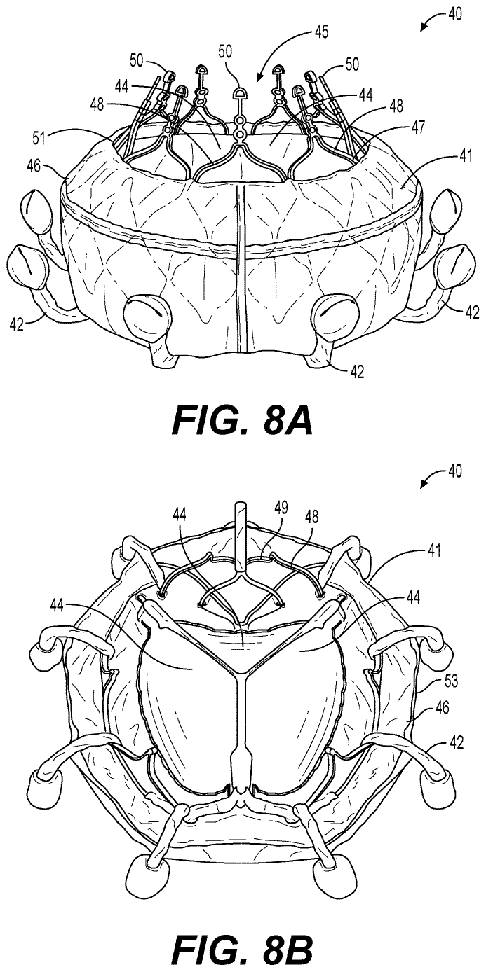

In still further aspects, the implantable device is a prosthetic valve configured to be deployed to a native valve of the heart. Exemplary prosthetic valves are shown in FIGS. 2A-2B and 8A-8B and described below in more detail.

Referring to FIG. 2A, in this aspect, the prosthetic valve 2000, comprises a frame 2002, having an upper region 2004, an intermediate region 2006, and a lower region 2008. The valve further comprises a plurality of prosthetic leaves 2020, an inner skirt 2016, and an outer skirt 2018.

In still further aspects, the implantable medical device has an inflow end 2040 and an outflow end 2030, and the one or more anchors 2010 are positioned at the outflow end of the prosthetic valve. The anchors 2010 shown in the FIG. 2A and in FIG. 2B are positioned within the disclosed herein woven articles. The anchors can be defined by a neck 2012, an arm 2013, and a tip 2014 (or head).

In still further aspects, the implantable medical device is configured to be deployed to a native valve of a heart, wherein the prosthetic valve further comprises the plurality of prosthetic valve leaflets as shown herein, wherein the one or more anchors are coupled to the plurality of prosthetic valve leaflets, and each is configured to anchor to a portion of the heart.

An exemplary anchor enclosed within the disclosed herein article is shown in FIG. 7. It can be seen that the anchor 7000 has a hook shape. Still further, the anchor includes a surface 59 facing radially inward and a surface facing radially outward 61. The anchor has a neck 7012, an arm 7013, and a head or a tip 7014. The anchor is inserted into the woven article 6000 and is positioned in the lumen 6003 of the tubular member such that the cushioning member 6006 is positioned around the tip 7014. The covering member 6010 is folded on the cushioning member, and some portions of the tubular member 6004. It is understood that the edge 7015 of the covering member, after it is folded, is sealed with other parts of the woven article. It is understood that this sealing can be done by any known in the art methods, for example, suturing, laser welding, ultrasonic welding, and the like.

In some aspects, each of the one or more anchors is configured to hook around a native valve leaflet, with the surface facing radially inward facing towards the native valve leaflet, and wherein the second thickness of the cushioning member disposed on the inward surface of the anchor is greater than the second thickness of the cushioning member disposed on the outward surface of the anchor.

As disclosed in detail herein, the implantable medical device can further comprise a sealing element or an outer skirt 2018 (FIG. 2A). The outer skirt or the sealing element can be used for a paravalvular leak seal.

In such exemplary and unlimiting aspects, a portion of the sealing element can comprise a textile having a 3-D honeycomb pattern. In such aspects, the textile can comprise a weave pattern substantially identical to the weave pattern of the cushioning member. In still further aspects, the textile can comprise a plurality of weft yarns and/or a plurality of warp yarns that are substantially identical to a plurality of weft yarns and/or a plurality of warp yarns of the cushioning member. As shown, for example, in FIG. 2A, the portion of the sealing element can extend circumferentially between adjacent anchors of the plurality of anchors. In still further aspects, the portion of the sealing element can be positioned as opposed to each of the plurality of anchors.

Additional examples of implantable medical devices are shown in FIGS. 8A and 8B. FIGS. 8A and 8B illustrate an example of a prosthetic valve 40 that can be utilized with the systems, apparatuses, and methods disclosed herein. The prosthetic valve 40 comprises a prosthetic heart valve and can be configured to be deployed to a native valve of a heart. The prosthetic valve 40 can include distal anchors 42 and a plurality of prosthetic valve leaflets 44 (more clearly shown in the bottom view of FIG. 8B). The prosthetic valve 40 can include a valve body 41 that can support the plurality of prosthetic valve leaflets 44. In some aspects, the valve body 41, can include an outer skirt 46 (or inner skirt, not shown) and can include one or more frames.

The outer skirt 46, surrounds an outer surface of the valve body 41 and contacts a portion of a native heart valve, such as native heart valve leaflets, to seal with such portions. A frame 49 (marked in FIG. 8B) of the valve body 41 can support the plurality of prosthetic valve leaflets 44. In this exemplary aspect, the frame 49 can comprise an inner frame that can be positioned radially inward of an outer frame 47 (marked in FIG. 8A). In such aspects, the outer frame 47 can surround the inner frame 49.

The outer frame 47 can comprise a portion of a sealing body that can include the outer skirt 46 for sealing with a portion of the native heart valve. The outer skirt 46 can reduce the possibility of leakage outside of the flow channel of the prosthetic valve (e.g., paravalvular leakage). It is understood that the shown herein valves are only exemplary, and other configurations frames or valve bodies are also contemplated.

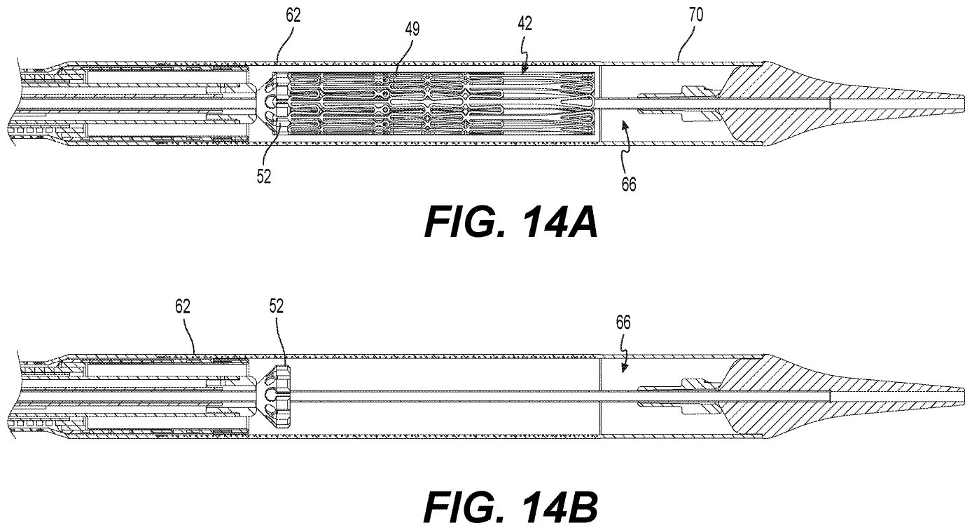

The implantable medical device can also include struts 48 that form the frame 49 of the prosthetic valve 40, and the outer frame 47 can include struts as well (FIGS. 8A-8B). Certain of the struts 48 can end in end tab portions 50 that are configured to couple to a portion of a delivery apparatus. For example, the end tab portion 50 (FIG. 8A) can be configured to engage a coupler 52 (as marked in FIGS. 14A and 14B) of a delivery apparatus to couple the prosthetic valve 40 to the delivery apparatus. The end tab portions 50 (FIG. 8A) can include flared half-dome shapes or another shape as desired to couple to the coupler 52. FIG. 8B illustrates a bottom view of the prosthetic valve 40.

The valve body 41 can surround a flow channel 45 (marked in FIG. 8A) that a central axis of the prosthetic valve 40 can extend along. The prosthetic valve leaflets 44 can extend radially inward from the valve body 41 towards the flow channel 45.

The plurality of prosthetic valve leaflets 44 can be configured to open and close to replicate the operation of a native valve. In the example shown in FIGS. 8A and 8B, the upper end 51 of the prosthetic valve 40 can comprise an inflow end of the prosthetic valve 40, and a lower end 53 of the prosthetic valve 40 can comprise an outflow end of the prosthetic valve 40. The prosthetic valve leaflets 44 can open and close to allow flow from the inflow end to the outflow end and impede flow from the outflow end to the inflow end.

The prosthetic valve 40 shown in FIGS. 8A and 8B is configured as a prosthetic mitral valve; however, other forms of implants can be utilized as desired. For example, prosthetic aortic, tricuspid, or pulmonary valves can be utilized with the systems, apparatuses, and methods disclosed herein. Further, other forms of implants, such as stents or other implants, can be utilized with the systems, apparatuses, and methods disclosed herein as well.

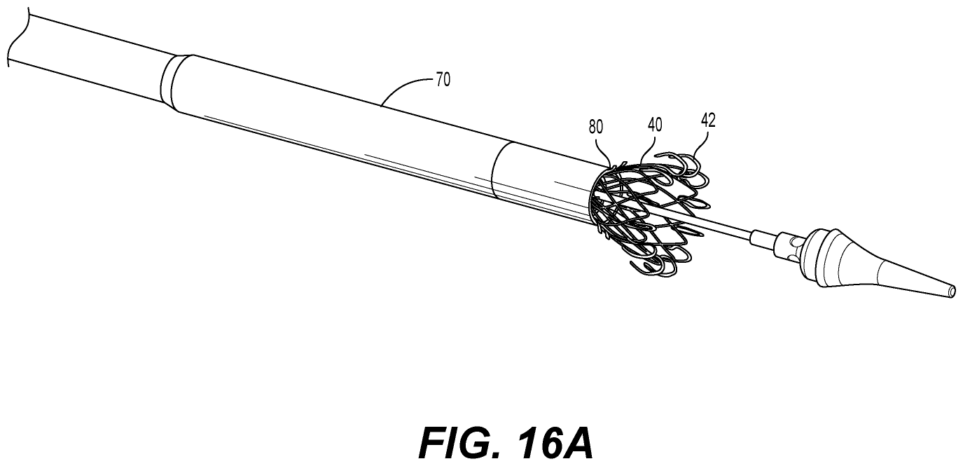

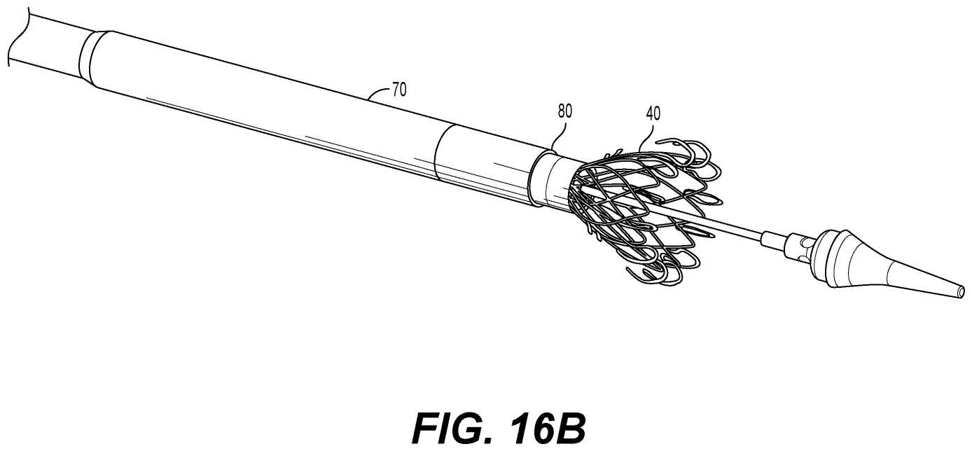

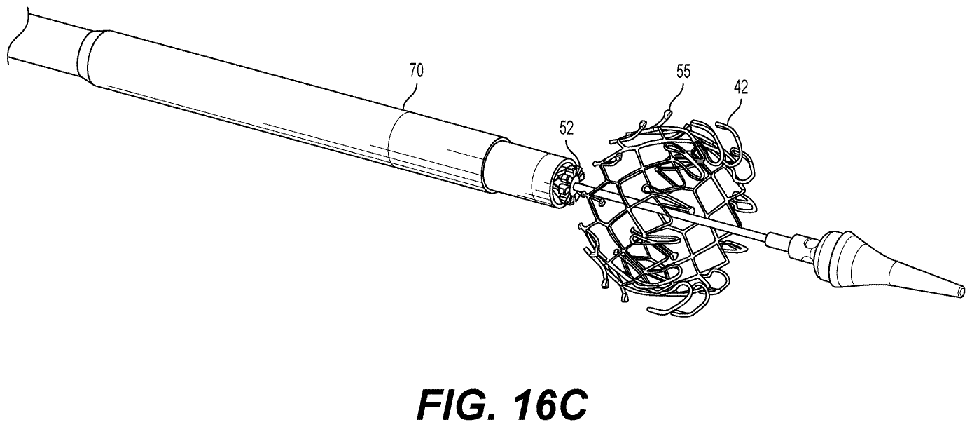

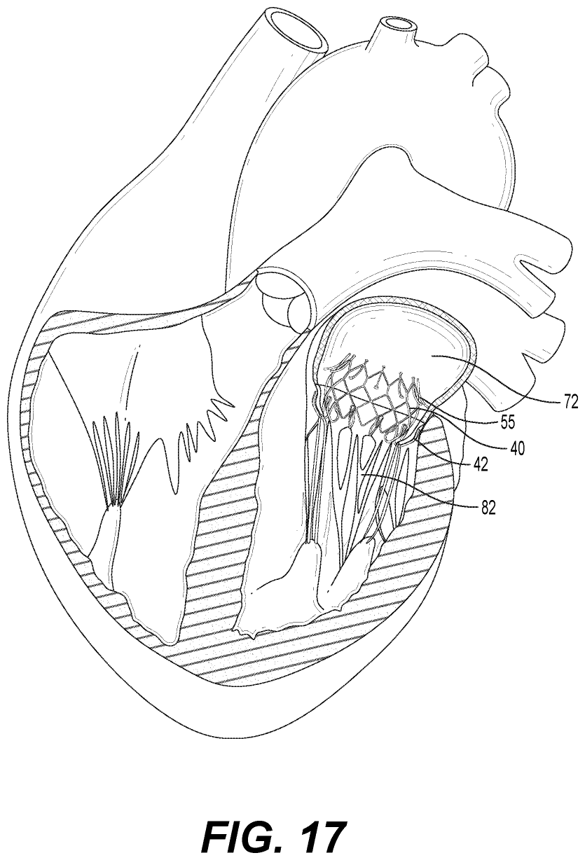

The prosthetic valve 40 shown in FIGS. 8A and 8B can utilize the distal anchors 42 to engage a ventricular side of a valve, such as a mitral or tricuspid valve. The distal anchors 42 can comprise ventricular anchors that are positioned at the outflow end of the prosthetic valve 40. The distal anchor 42 can have a hook shape that allows each distal anchor 42 to hook around a native valve leaflet to secure the prosthetic valve 40 to the native valve. Each anchor 42 can comprise an elongated arm having a length. In some aspects, the prosthetic valve 40 can also include proximal anchors 55 that can engage an atrial side of a mitral valve, as marked in FIGS. 16C and 17. It is understood that in some aspects, such anchors 55 are present, while in other aspects, they are excluded. One or more anchors can be coupled to the plurality of prosthetic valve leaflets 44 and each configured to anchor to a portion of the heart. Other forms of anchors can be utilized, as well as other forms of implants can be utilized in other aspects.

Features of an implant that can be utilized are disclosed in U.S. patent application Ser. No. 16/028,172, the entire content of which is incorporated by reference herein. Additional details and example designs for an implant and prosthesis that can be utilized in examples herein are described in U.S. Pat. Nos. 8,403,983, 8,414,644, 8,652,203 and U.S. Patent Publication Nos. 2011/0313515, 2012/0215303, 2014/0277390, 2014/0277422, 2014/0277427, 2018/0021129, and 2018/0055629, the entireties of these patents and publications are hereby incorporated by reference for all purposes and made a part of this specification. Further details and examples of a replacement heart valve or prosthesis and its method of implantation are described in U.S. Publication Nos. 2015/0328000 and 2016/0317301, the entirety of each of which is hereby incorporated by reference and made a part of this specification.

The prosthetic valve 40 is compressible, such that the prosthetic valve 40 can be compressed in a direction radially towards a longitudinal axis or central axis that the prosthetic valve 40 surrounds. For example, the frame of the prosthetic valve 40 can be flexible to allow for compression of the prosthetic valve 40. Upon compression in the direction radially towards the longitudinal axis, the prosthetic valve 40 can increase in length longitudinally along the longitudinal axis (with the end tab portions 50 and the distal anchors 42 extending in opposite directions along the longitudinal axis, for example, as shown in FIG. 14A). In certain examples, the prosthetic valve 40 can be configured to compress in the direction radially towards the longitudinal axis without the prosthetic valve 40 increasing in length.

The anchors 42, as shown in FIGS. 8A and 8B are inserted into the disclosed herein woven articles, wherein cushioning members of the articles help cushion the anchors 42 against the native structure of the patient's heart. The anchors 42, for example, can comprise a rigid structure. In some aspects, anchors 42 can be coupled to the frame 49 and integral with the struts 48 of the frame 49. Similar to disclosed anchors above, the anchors 42 can include arms in the form of hooks that are rigid and can damage the native structure of the patient's heart if they directly contact the native structure. The disclosed herein woven articles cover the anchors 42 to protect the native structure from such contact.

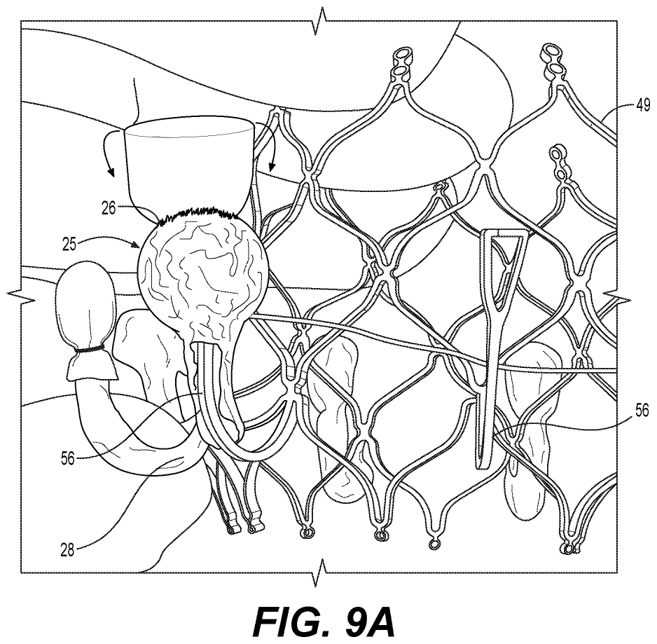

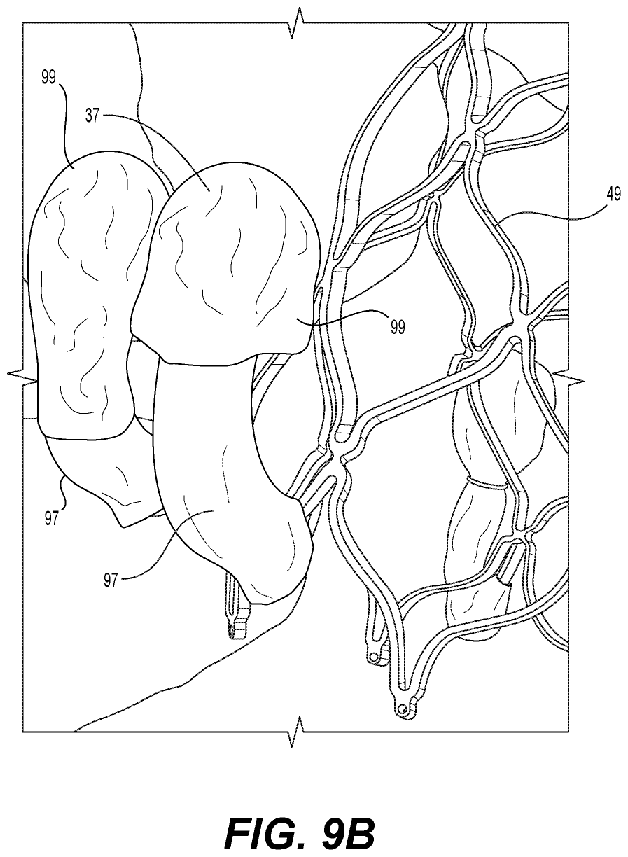

FIG. 9A illustrates a step in a process of coupling the woven article 25 to an arm 56 of an anchor 42. The “cut-out” of the article is shown to demonstrate that arm 56 is positioned within the article. The neck portion 28 is positioned within the tubular member of the article, whereas the cushioning member 26 is positioned around the tip of the anchor. The covering member is shown prior to being folded on the cushioning member.

FIG. 9B shows an exemplary assembled woven article on the anchors 42 on the frame 49. It can be seen in this exemplary aspect that the cushioning member 37 covers the head of the anchor, and the tubular member 97 covers the arm and the neck of the anchor. The covering member 99 is shown folded and then attached to other parts of the woven article.

The woven articles of this disclosure provide improved compressibility to cushion the anchor 42. Such improved compressibility can be desired over prior forms of cushions that can include foam or another closed-cell material. The improved compressibility can further provide benefits during the deployment of the prosthetic valve 40.

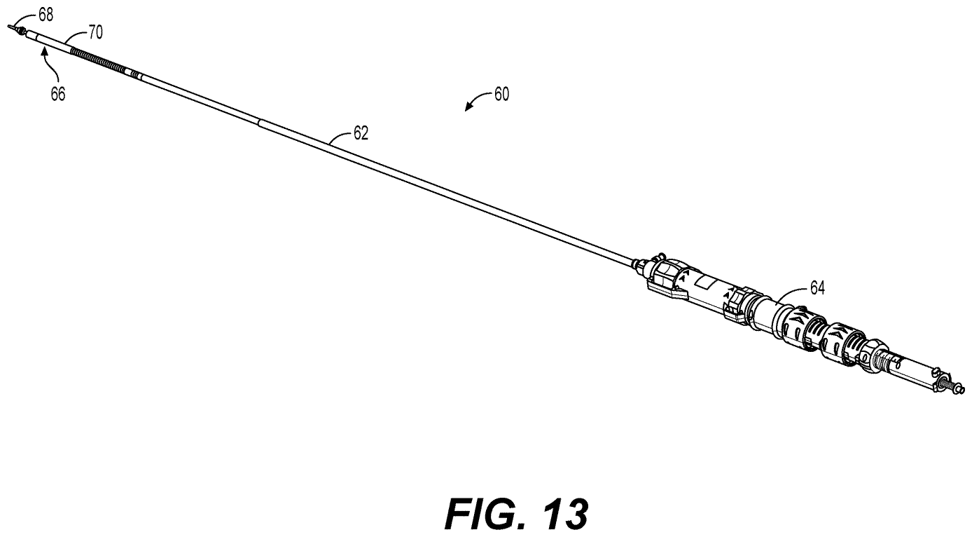

FIG. 13, for example, illustrates an aspect of a delivery apparatus 60 that can be used according to examples herein to deploy an implant, such as the prosthetic valve 40. The delivery apparatus 60 can include an elongated shaft 62 having a proximal end coupled to a handle 64 and a distal end, including an implant retention area 66 for the prosthetic valve 40, a nose cone 68, and a capsule 70 that extends over the implant retention area 66. The elongated shaft 62 can be deflectable to position the prosthetic valve 40 in a desired position relative to an implantation site. Features of a delivery apparatus that can be utilized, as well as methods of deployment using a delivery apparatus, are disclosed in U.S. patent application Ser. No. 16/028,172, filed on Jul. 5, 2018, and published as U.S. Patent Publication No. 2019/0008640, the entire contents of which are incorporated by reference herein.

[The prosthetic valve 40 can be crimped in an elongated configuration within the capsule 70 prior to deployment, as shown in FIG. 14A, for example. Only the frame 49 of the prosthetic valve 40 is shown in FIG. 14A for clarity. The distal anchors 42 are compressed and extend longitudinally along the longitudinal axis or central axis of the elongated shaft 62. The force of compression of the capsule 70 upon the distal anchors 42 is preferably low to reduce the deployment force needed to deploy the prosthetic valve 40 from the capsule 70.

FIG. 14B illustrates the prosthetic valve 40 absent from the elongated shaft 62.

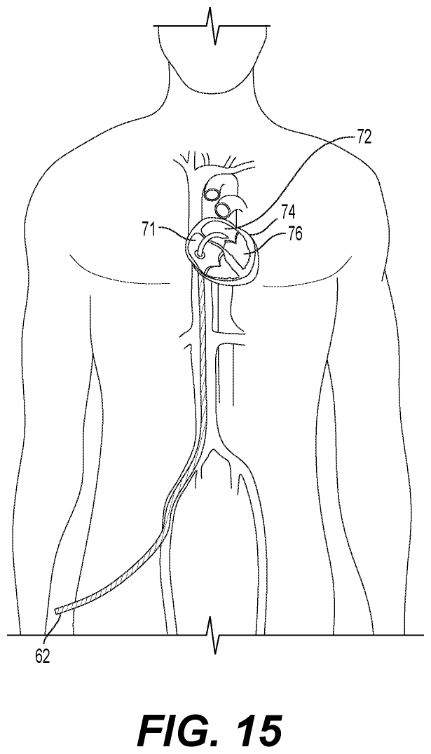

Deployment of the prosthetic valve 40 can occur according in a variety of forms, including transfemoral entry or surgical methods of entry into the patient's heart. FIG. 15 illustrates an exemplary procedure in which femoral entry is utilized. The elongated shaft 62 of the delivery apparatus 60 can pass through the femoral vein, among other entry points. FIG. 15 illustrates the elongated shaft 62 of the delivery apparatus 60 passing transfemoral to a right atrium 71 and then transseptal to the left atrium 72 of the patient's heart 74. The elongated shaft 62 can deflect to position the prosthetic valve 40 for deployment to the mitral valve. The elongated shaft 62 can deflect towards the left ventricle 76.

FIGS. 16A-16C illustrate steps in the deployment of the prosthetic valve 40, in which the capsule 70 retracts relative to the prosthetic valve 40. The prosthetic valve 40 is deployed to a native valve of a patient's heart. The prosthetic valve 40 extends out of an opening 80 in the capsule 70. The distal anchors 42 can extend distally and can then deflect proximally to hook around native valve leaflets upon deployment. Each of the distal anchors 42 can be hooked around a native valve leaflet. The disclosed herein woven articles (not shown in FIGS. 16A-16C) can cushion the anchors 42 upon deployment, and the compressibility of the pads can reduce the deployment force of the prosthetic valve 40.

FIGS. 16B and 16C illustrate the capsule 70 further retracting to deploy the prosthetic valve 40.

FIG. 17 illustrates the prosthetic valve 40 deployed to a native mitral valve, with the distal anchors 42 hooked around the native valve leaflets. The distal anchors 42 can hook around the native valve leaflets and can hook around the chordae 82 in examples if desired.

Other deployment methods can be utilized in some aspects. For example, balloon-expandable prosthetic valves can be utilized. Such valves can be positioned upon an inflatable balloon upon entry into a patient's body or can be slid onto an inflatable balloon following entry into the patient's body. The inflatable balloon can be inflated with fluid to expand and deploy the prosthetic valve. Mechanically expandable prosthetic valves can further be utilized, along with self-expanding valves and other forms of deployment.

In some aspects, the prosthetic valve 40 can be deployed to other valves, such as the tricuspid valve in examples. Other forms of prosthetic valves can be utilized in the examples herein to deploy to other valves, such as the aortic or pulmonary valves, or to other portions of the patient's body.

The implantable medical devices disclosed herein can comprise a mitral replacement valve or a tricuspid replacement valve, among other forms of valves (e.g., aortic replacement valves, pulmonary replacement valves, or other valves). The implantable medical devices disclosed herein can include prosthetic heart valves or other forms of implants, such as stents filters or diagnostic devices, among others. The implantable medical devices can be expandable implants configured to move from a compressed or undeployed state to an expanded or deployed state. The implantable medical devices can be compressible implants configured to be compressed inward to have a reduced outer profile and to move the implant to the compressed or undeployed state.

Various forms of delivery apparatuses can be utilized with the examples disclosed herein. The delivery apparatuses as disclosed herein can be utilized for aortic, mitral, tricuspid, and pulmonary replacement and repair as well. The delivery apparatuses can comprise delivery apparatuses for the delivery of other forms of implants, such as stent filters or diagnostic devices, among others.

The implantable medical devices and the systems disclosed herein can be used in transcatheter aortic valve implantation (TAVI) or replacement of other native heart valves (e.g., mitral, tricuspid, or pulmonary). The delivery apparatuses and the systems disclosed herein can be utilized for transarterial access, including transfemoral access, to a patient's heart. The delivery apparatuses and systems can be utilized in transcatheter percutaneous procedures, including transarterial procedures, which can be transfemoral or transjugular. Transapical procedures, among others, can also be utilized. Other procedures can be utilized as desired. Features of aspects can be modified, substituted, excluded, or combined across examples as desired.

### Methods

The present disclosure also provides for a method comprising weaving any of the disclosed herein woven articles to form a 3-D unibody article. The article is woven on known in the art equipment. In still further aspects, the weave of each member is done in a separate plane, simultaneously creating the desired 3-D unibody article. In still further aspects, the woven article disclosed herein can be exposed to heat-treatment conditions at a predetermined temperature and time, where the heat-treatment conditions are effective in providing the desired mechanical properties of each member. It is understood that the article can be exposed to heat treatment when the entire article is formed or at any step of the weaving process as desired.

In still further aspects, also disclosed herein are methods of making the disclosed herein implantable medical device, wherein the method comprises providing a device comprising one or more anchors, wherein each of the one or more anchors is configured to anchor the implantable medical device to a portion of a patient's natural anatomy, and inserting each anchor into the disclosed woven article.

Still further disclosed is a method comprising: deploying a prosthetic valve to a native valve of a patient's heart, the prosthetic valve comprising: a plurality of prosthetic valve leaflets, one or more anchors coupled to the plurality of prosthetic valve leaflets, and each configured to anchor to a portion of the patient's heart, wherein each of the one or more anchors is inserted into the disclosed herein woven article. In such aspects, the one or more anchors comprise ventricular anchors. In still further aspects, the method further comprises hooking each of the one or more anchors around a native valve leaflet. In still further aspects, the native valve is a native mitral valve or a native tricuspid valve.

### Exemplary Aspects

In view of the described processes and compositions, hereinbelow are described certain more particularly described aspects of the disclosures. These particularly recited aspects should not, however, be interpreted to have any limiting effect on any different claims containing different or more general teachings described herein, or that the “particular” aspects are somehow limited in some way other than the inherent meanings of the language and formulas literally used therein.
Example 1: A woven articled defined by a proximal end and a distal end, wherein the woven article is a 3-D unibody article and comprises: a) a tubular member having a lumen disposed between a first open end of the tubular member and a second end of the tubular member; wherein the tubular member has a first thickness, wherein the first open end defines the proximal end of the woven article; wherein the lumen has a diameter d₁ and a length l₁; b) a cushioning member having a second thickness, wherein an outer surface of the tubular member seamlessly transitions into an outer surface of the cushioning member at a length l₂ from the first open end of the tubular member, thereby forming a proximal end of the cushioning member, wherein l₂ is less than l₁, wherein a portion of the cushioning member comprises a central cavity housing a portion of the lumen having a length l₃, such that the second end of the tubular member is positioned within the central cavity of cushioning member; and c) a woven seal positioned at a distal end of the cushioning member.
Example 2: The woven article of any examples herein, particularly example 1, wherein the length l₃ is equal to l₁−l₂.
Example 3: The woven article of any examples herein, particularly example 1 or 2, wherein the cushioning member has a regular or irregular shape.
Example 4: The woven article of any examples herein, particularly example 3, wherein the cushioning member has a spherical, cube, cuboid, pyramidal, oval, cone, or prism shape.
Example 5: The woven article of any examples herein, particularly example 3 or 4, wherein the cushioning member has a regular or irregular cross-sectional shape.
Example 6: The woven article of any examples herein, particularly example 5, wherein the cushioning member has a circular, oval, elliptical, rectangular, square, cross-sectional shape, or any combination thereof.
Example 7: The woven article of any examples herein, particularly examples 5-6, wherein the cross-sectional shape is defined by a length a₁ along at least one of the x- or y-axis.
Example 8: The woven article of any examples herein, particularly examples 1-7, wherein the woven article comprises a plurality of warp yarns and weft yarns.
Example 9: The woven article of any examples herein, particularly examples 1-8, wherein the tubular member has a plain weave pattern, a twill weave, a satin weave, any derivative, or any combination thereof.
Example 10: The woven article of any examples herein, particularly examples 1-9, wherein the cushioning member has a 3-D honeycomb pattern having a plurality of repeating cell units.
Example 11: The woven article of any examples herein, particularly example 10, wherein the plurality of repeating cell units comprise from about 1 to about 32 cells per inch.
Example 12: The woven article of any examples herein, particularly example 10 or 11, wherein each of the plurality of repeating cell units comprises about 8 to 50 weft yarn insertion repeats per linear inch.
Example 13: The woven article of any examples herein, particularly examples 1-12, wherein the tubular member transitions into the cushioning member at the proximal end of the cushioning member such that a first tapered-out portion of the cushioning member is formed.
Example 14: The woven of any examples herein, particularly example 13, wherein the first tapered-out portion is formed by increasing a spacing between a plurality of warp yarns during a weft yarn insertion in a weaving process.
Example 15: The woven article of any examples herein, particularly examples 13-14, wherein the first tapered-out portion is formed by adding at least one warp yarn during a weaving process.
Example 16: The woven article of any examples herein, particularly examples 1-15, wherein a distal portion of the cushioning member comprises a first tapered-in portion.
Example 17: The of any examples herein, particularly example 16, wherein the first tapered-in portion terminates in the distal end of the cushioning member, wherein the distal end of the cushioning member transitions into the woven seal.
Example 18: The woven of any examples herein, particularly example 16 or 17, wherein the first tapered-in portion is formed by decreasing a spacing between a plurality of warp yarns during a weft yarn insertion in a weaving process.
Example 19: The woven article of any examples herein, particularly examples 16-18, wherein the first tapered-in portion is formed by removing at least one warp yarn during a weaving process.
Example 20: The woven article of any examples herein, particularly examples 16-19, wherein the first tapered-in portion is terminated by the woven seal.
Example 21: The woven article of any examples herein, particularly examples 1-20, wherein the first thickness is smaller than the second thickness.
Example 22: The woven article of any examples herein, particularly examples 1-21, wherein the first thickness is the same or different along a circumference of the tubular member and/or along the length l₂.
Example 23: The woven article of any examples herein, particularly examples 1-22, wherein the second thickness is the same or different along a circumference and/or a perimeter of the cushioning member and/or along a length of the cushioning member.
Example 24: The woven article of any examples herein, particularly examples 1-23, wherein the cushioning member is defined by a cushioning gradient.
Example 25: The woven article of any examples herein, particularly example 24, wherein the cushioning gradient is achieved by varying the weave density, varying the quantity of repeating cell units, varying a number of the weft yarn insertions in the plurality of weft yarns, varying the denier of the weft yarns, varying a texture of the weft yarns, or by a combination thereof.
Example 26: The woven article of any examples herein, particularly examples 1-25, wherein the l₂ is equal to or greater than 10 mm.
Example 27: The woven article of any examples herein, particularly examples 1-26, wherein d₁ is about 2.5 mm to about 3.5 mm.
Example 28: The woven article of any examples herein, particularly examples 7-27, wherein a₁ is about 3 mm to about 5 mm.
Example 29: The woven article of any examples herein, particularly examples 13-28, wherein the first tapered-out portion is about 0.5 mm to about 1.5 mm.
Example 30: The woven article of any examples herein, particularly examples 17-29, wherein the first tapered-in portion is about 0.5 mm to about 1.5 mm.
Example 31: The woven article of any examples herein, particularly examples 1-30, wherein the woven seal has a length in a longitudinal direction of about 1 mm to about 3 mm.
Example 32: The woven article of any examples herein, particularly examples 1-31, wherein the second end of the tubular member positioned within the central cavity of the cushioning member is closed.
Example 33: The woven article of any examples herein, particularly examples 1-32, wherein the portion of the lumen positioned within the central cavity of the cushioning member behaves as an inner pocket of the article.
Example 34: The woven article of any examples herein, particularly examples 1-33, further comprises a covering member having a third thickness and is defined by an inner surface and an outer surface and having a proximal end and a distal end, wherein the proximal end of the covering member is coupled to the woven seal and wherein the distal end is open.
Example 35: The woven article of any examples herein, particularly example 34, wherein the third thickness is the same or different along a circumference and/or a perimeter of the covering member and/or along a length of the covering member.
Example 36: The woven article of any examples herein, particularly example 35 or 36, wherein the coupling between the woven seal and the proximal end of the covering member is formed during a weaving process.
Example 37: The woven article of any examples herein, particularly examples 34-36, wherein the covering member comprises a second tapered-out portion positioned outwards and up from the proximal end of the covering member.
Example 38: The woven article of any examples herein, particularly examples 34-37, wherein the covering member has a regular or irregular shape.
Example 39: The woven article of any examples herein, particularly example 38, wherein the covering member has a length a₂ along at least one of the x- or y-axis, wherein a₂ is greater than a₁.
Example 40: The woven article of any examples herein, particularly examples 34-39, wherein the covering member has a plain weave pattern, a twill weave, a satin weave, any derivative, or any combination thereof weave pattern.
Example 41: The woven article of any examples herein, particularly examples 34-40, wherein the covering member comprises a second tapered-in portion at the distal portion of the covering member, wherein the second tapered-in portion terminates in the open distal end of the covering member having a length a₃ along at least one of x or y-axis.
Example 42: The woven article of any examples herein, particularly example 41, wherein a₃ is smaller than a₂ and equal to or greater than a₁.
Example 43: The woven article of any examples herein, particularly examples 34-42, wherein the covering member is configured to be folded over the cushioning member such that the outer surface of the covering member is positioned above the outer surface of the cushioning member and the inner surface of the covering member is exposed to a surrounding environment.
Example 44: The woven article of any examples herein, particularly examples 8-43, wherein the plurality of warp yarns and the plurality of weft yarns comprise a biocompatible material.
Example 45: The woven article of any examples herein, particularly examples 8-44, wherein the plurality of warp yarns are the same or different from the plurality of weft yarns.
Example 46: The woven article of any examples herein, particularly examples 8-45, wherein at least one of the plurality of warp yarns and/or a plurality of weft yarns comprise one or more biocompatible textured multifilament yarn, a biocompatible textured high shrinkage multifilament yarn, a biocompatible flat multifilament yarn, a twisted multifilament yarn, or any combination thereof.
Example 47: The woven article of any examples herein, particularly examples 8-46, wherein the plurality of warp yarns and a plurality of weft yarns comprise polyesters, aliphatic polyesters, co-polyesters, polyolefins, fluoropolymers, polyurethanes, polyethers, polyureas, nylons, natural fibers, copolymers thereof, or a combination thereof.
Example 48: The woven article of any examples herein, particularly example 47, wherein the plurality of warp yarns and a plurality of weft yarns comprise ultra-high molecular weight polyethylene, polyethylene, polypropylene, polytetrafluoroethylene, expanded polytetrafluorethylene, polyvinylidene fluoride, or any combination thereof.
Example 49: The woven article of any examples herein, particularly examples 8-48, wherein the plurality of warp yarns and a plurality of weft yarns comprise non-resorbable yarns, bio-resorbable yarns, or a combination thereof.
Example 50: The woven article of any examples herein, particularly example 49, wherein the bio-resorbable yarns comprise one or more aliphatic polyester or co-polyesters, or a combination thereof.
Example 51: The woven article of any examples herein, particularly example 50, wherein the bio-resorbable yarns comprise polyglycolic acid (PGA), poly(L-lactic acid (PLLA), polycaprolactone (PCL), polylactide glycolide copolymers (PLGA), copolymers thereof, or a combination thereof.
Example 52: The woven article of any examples herein, particularly examples 1-51, wherein the woven article is configured to be assembled in an implantable medical device comprising: one or more anchors, wherein each of the one or more anchors is configured to anchor the implantable medical device into a patient's natural anatomy.
Example 53: The woven of any examples herein, particularly example 52, wherein the woven article is configured to receive an anchor of the one or more anchors through the first open end of the tubular member such that the anchor is positioned within the lumen of the tubular member, such that the second end of the tubular member substantially prevent a further anchor's movement within the article.
Example 54: The woven of any examples herein, particularly example 52 or 53, wherein the cushioning member protects the patient's natural anatomy.
Example 55: The woven article of any examples herein, particularly examples 34-54, wherein the covering member, if present, is configured to form a substantially smooth surface that is exposed to the patient's natural anatomy.
Example 56: The woven article of any examples herein, particularly examples 1-55, wherein the article is configured to promote a tissue response.
Example 57: The woven article of any examples herein, particularly examples 1-56, wherein the woven article comprises one or more apertures disposed circumferentially in predetermined locations, wherein the one or more apertures are configured to receive a fastening member for an attachment of the woven article to a substrate.
Example 58: An implantable medical device comprising one or more anchors, wherein each of the one or more anchors is configured to anchor the implantable medical device to a portion of a patient's natural anatomy, wherein each of the anchors is positioned within the woven article of any examples herein, particularly examples 1-57.
Example 59: The implantable medical device of any examples herein, particularly example 58, wherein the implantable device is a prosthetic valve configured to be deployed to a native valve of a heart, wherein the valve further comprises a plurality of prosthetic valve leaflets, wherein the one or more anchors are coupled to the plurality of prosthetic valve leaflets, and each is configured to anchor to a portion of the heart.
Example 60: The implantable medical device of any examples herein, particularly example 59, wherein the prosthetic valve includes an inflow end and an outflow end, and the one or more anchors are positioned at the outflow end of the prosthetic valve.
Example 61: The implantable medical device of any examples herein, particularly example 58 or 59, wherein the one or more anchors comprise ventricular anchors.
Example 62: The implantable medical device of any examples herein, particularly examples 58-61, wherein each of the one or more anchors has a hook shape.
Example 63: The implantable medical device of any examples herein, particularly examples 59-62, wherein each of the one or more anchors includes a surface facing radially inward and a surface facing radially outward.
Example 64: The implantable medical device of any examples herein, particularly example 63, wherein each of the one or more anchors is configured to hook around a native valve leaflet, with the surface facing radially inward facing towards the native valve leaflet, and wherein the second thickness of the cushioning member disposed on the inward surface of the anchor is greater than the second thickness of the cushioning member disposed on the outward surface of the anchor.
Example 65: The implantable medical device of any examples herein, particularly examples 59-64, further comprising a frame supporting the plurality of prosthetic valve leaflets and wherein the one or more anchors are coupled to a frame.
Example 66: The implantable medical device of any examples herein, particularly examples 59-65, further comprising a sealing element secured to a frame, wherein a portion of the sealing element comprises: a textile having a 3-D honeycomb pattern.
Example 67: The implantable medical device of any examples herein, particularly example 66, wherein the textile comprises a weave pattern substantially identical to a weave pattern of the cushioning member.
Example 68: The implantable medical device of any examples herein, particularly example 66 or 67, wherein the textile comprises a plurality of weft yarns and/or a plurality of warp yarns that are substantially identical to a plurality of weft yarns and/or a plurality of warp yarns of the cushioning member
Example 69: The implantable medical device of any examples herein, particularly examples 66-68, wherein the portion of the sealing element extends circumferentially between adjacent anchors of the plurality of anchors.
Example 70: The implantable medical device of any examples herein, particularly examples 66-69, wherein the portion of the sealing element is positioned opposed to each of the plurality of anchors.
Example 71: An implantable device comprising a self-expanding frame having an upper region, an intermediate region and a lower region, wherein the frame is configured to radially expand and contract for deployment within a body cavity, and wherein the self-expanding frame comprises one or more anchors disposed along the lower region; wherein each of the one or more anchors is configured to anchor the implantable medical device to a portion of a patient's natural anatomy, and wherein at least a portion of the one or more anchors is positioned within a woven article; wherein the woven article comprises: a tubular member having a lumen disposed between a first open end of the tubular member and a second end of the tubular member; wherein the tubular member has a first thickness, wherein the first open end defines the proximal end of the woven article; wherein the lumen has a diameter d₁ and a length l₁; a cushioning member having a second thickness, wherein an outer surface of the tubular member seamlessly transitions into an outer surface of the cushioning member at a length l₂ from the first open end of the tubular member, thereby forming a proximal end of the cushioning member, wherein l₂ is less than l₁, wherein a portion of the cushioning member comprises a central cavity housing a portion of the lumen having a length l₃, such that the second end of the tubular member is positioned within the central cavity of cushioning member; and a woven seal positioned at a distal end of the cushioning member.
Example 72: A method comprising weaving the woven article of any examples herein, particularly examples 1-57, to form a 3-D unibody article.
Example 73: A method of making the implantable medical device of any examples herein, particularly examples 58-71, wherein the method comprises: a) providing a device comprising one or more anchors, wherein each of the one or more anchors is configured to anchor the implantable medical device to a portion of a patient's natural anatomy, and b) inserting each anchor into the woven article of any examples herein, particularly examples 1-57.
Example 74: A method comprising: deploying a prosthetic valve to a native valve of a patient's heart, the prosthetic valve comprising: a plurality of prosthetic valve leaflets, one or more anchors coupled to the plurality of prosthetic valve leaflets, and each configured to anchor to a portion of the patient's heart, wherein each of the one or more anchors is inserted into the woven article of any examples herein, particularly examples 1-57.
Example 75: The method of any examples herein, particularly example 74, wherein the one or more anchors comprise ventricular anchors.
Example 76: The method of any examples herein, particularly example 74 or 75, further comprising hooking each of the one or more anchors around a native valve leaflet.
Example 77: The method of any examples herein, particularly examples 74-76, wherein the native valve is a native mitral valve or a native tricuspid valve.

Although several aspects of the disclosure have been disclosed in the foregoing specification, it is understood by those skilled in the art that many modifications and other aspects of the disclosure will come to mind to which the disclosure pertains, having the benefit of the teaching presented in the foregoing description and associated drawings. Thus, it is understood that the disclosure is not limited to the specific aspects disclosed hereinabove and that many modifications and other aspects are intended to be included within the scope of the appended claims. Moreover, although specific terms are employed herein, as well as in the claims which follow, they are used only in a generic and descriptive sense and not for the purposes of limiting the described disclosure nor the claims which follow. Therefore, we claim as our disclosure all that comes within the scope and spirit of these claims.

We claim:

1. A woven article defined by a proximal end and a distal end, wherein the woven article is a 3-D unibody article and comprises:
(a) a tubular member having a lumen disposed between a first open end of the tubular member and a second end of the tubular member; wherein the tubular member has a first thickness,
wherein the first open end defines the proximal end of the woven article;
wherein the lumen has a diameter d1 and a length l1;
(b) a cushioning member having a second thickness,
wherein an outer surface of the tubular member seamlessly transitions into an outer surface of the cushioning member at a length l₂ from the first open end of the tubular member, thereby forming a proximal end of the cushioning member, wherein l2 is less than l1,
wherein a portion of the cushioning member comprises a central cavity housing a portion of the lumen having a length l₃, such that the second end of the tubular member is positioned within the central cavity of the cushioning member; and
(c) a woven seal positioned at a distal end of the cushioning member.

2. The woven article of claim 1, wherein the length l₃ is equal to l₁−l₂.

3. The woven article of claim 1, wherein the cushioning member has a regular or irregular shape and/or a regular or irregular cross-sectional shape.

4. The woven article of claim 3, wherein the cross-sectional shape is defined by a length a1 along at least one of the x- or y-axis.

5. The woven article of claim 1, wherein the tubular member has a plain weave pattern, a twill weave, a satin weave, any derivative, or any combination thereof.

6. The woven article of claim 1, wherein the cushioning member has a 3-D honeycomb pattern having a plurality of repeating cell units, such that the plurality of repeating cell units comprise from about 1 to about 32 cells per inch and/or wherein each of the plurality of repeating cell units comprises about 8 to 50 weft yarn insertion repeats per linear inch.

7. The woven article of claim 1, wherein the tubular member transitions into the cushioning member at the proximal end of the cushioning member such that a first tapered-out portion of the cushioning member is formed.

8. The woven article of claim 7, wherein the first tapered-out portion is formed by increasing a spacing between a plurality of warp yarns during a weft yarn insertion in a weaving process and/or wherein the first tapered-out portion is formed by adding at least one warp yarn during a weaving process.

9. The woven article of claim 1, wherein a distal portion of the cushioning member comprises a first tapered-in portion, wherein the first tapered-in portion terminates in the distal end of the cushioning member, wherein the distal end of the cushioning member transitions into the woven seal; and/or
wherein the first tapered-in portion is formed by decreasing a spacing between a plurality of warp yarns during a weft yarn insertion in a weaving process; and/or
wherein the first tapered-in portion is formed by removing at least one warp yarn during a weaving process; and/or
wherein the first tapered-in portion is terminated by the woven seal.

10. The woven article of claim 1, wherein the first thickness is smaller than the second thickness.

11. The woven article of claim 1, wherein the first thickness is the same or different along a circumference of the tubular member and/or along the length l2; and/or wherein the second thickness is the same or different along a circumference and/or a perimeter of the cushioning member and/or along a length of the cushioning member.

12. The woven article of claim 1, wherein the cushioning member is defined by a cushioning gradient that is achieved by varying a weave density, varying a quantity of repeating cell units, varying a number of a weft yarn insertions in a plurality of weft yarns, varying a denier of the weft yarns, varying a texture of the weft yarns, or by a combination thereof.

13. The woven article of claim 1, wherein the second end of the tubular member positioned within the central cavity of the cushioning member is closed; and/or wherein the portion of the lumen positioned within the central cavity of the cushioning member behaves as an inner pocket of the article.

14. The woven article of claim 1, further comprises a covering member having a third thickness and is defined by an inner surface and an outer surface and having a proximal end and a distal end, wherein the proximal end of the covering member is coupled to the woven seal and wherein the distal end is open and wherein the third thickness is the same or different along a circumference and/or a perimeter of the covering member and/or along a length of the covering member.

15. The woven article of claim 14, wherein the coupling between the woven seal and the proximal end of the covering member is formed during a weaving process.

16. The woven article of claim 14, wherein the covering member comprises a second tapered-out portion positioned outwards and up from the proximal end of the covering member, wherein the covering member has a regular or irregular shape; and the covering member has a length a₂ along at least one of the x- or y-axis, wherein a₂ is greater than a₁.

17. The woven article of claim 14, wherein the covering member comprises a second tapered-in portion at the distal portion of the covering member, wherein the second tapered-in portion terminates in the open distal end of the covering member having a length a₃ along at least one of the x- or y-axis; and wherein a₃ is smaller than a₂ and equal to or greater than a₁.

18. The woven article of claim 14, wherein the covering member is configured to be folded over the cushioning member such that the outer surface of the covering member is positioned above the outer surface of the cushioning member and the inner surface of the covering member is exposed to a surrounding environment.

19. The woven article of claim 1, wherein the article is configured to be assembled in an implantable medical device comprising:
one or more anchors, wherein each of the one or more anchors is configured to anchor the implantable medical device into a patient's natural anatomy, such that the woven article is configured to receive an anchor of the one or more anchors through the first open end of the tubular member such that the anchor is positioned within the lumen of the tubular member, such that the second end of the tubular member substantially prevent a further anchor's movement within the article.

20. An implantable medical device comprising one or more anchors, wherein each of the one or more anchors is configured to anchor the implantable medical device to a portion of a patient's natural anatomy, wherein each of the anchors is positioned within the woven article of claim 1.

21. The implantable medical device of claim 20, further comprising a sealing element secured to a frame, wherein a portion of the sealing element comprises: a textile having a 3-D honeycomb pattern.

22. An implantable medical device comprising a self-expanding frame having an upper region, an intermediate region, and a lower region, wherein the frame is configured to radially expand and contract for deployment within a body cavity and wherein the self-expanding frame comprises one or more anchors disposed along the lower region;
wherein each of the one or more anchors is configured to anchor the implantable medical device to a portion of a patient's natural anatomy, and wherein at least a portion of the one or more anchors is positioned within a woven article; wherein the woven article comprises:
a tubular member having a lumen disposed between a first open end of the tubular member and a second end of the tubular member;
wherein the tubular member has a first thickness,
wherein the first open end defines a proximal end of the woven article;
wherein the lumen has a diameter d₁ and a length l₁;
a cushioning member having a second thickness,
wherein an outer surface of the tubular member seamlessly transitions into an outer surface of the cushioning member at a length l₂ from the first open end of the tubular member, thereby forming a proximal end of the cushioning member, wherein l₂ is less than l₁,
wherein a portion of the cushioning member comprises a central cavity housing a portion of the lumen having a length l₃, such that the second end of the tubular member is positioned within the central cavity of the cushioning member; and
a woven seal positioned at a distal end of the cushioning member.

23. A method comprising weaving the woven article of claim 1 to form a 3-D unibody article.
